(19) United States
(12) Patent Application Publication
(10) Pub. No.: US 2024/0065213 A1
(43) Pub. Date: Feb. 29, 2024

(54) SOYBEAN PLANT WITH HEALTHIER PROPERTIES

(71) Applicant: BETTERSEEDS LTD, Givat Chen (IL)

(72) Inventors: Ido MARGALIT, Gan Yavne (IL); Tal SHERMAN, Rechovot (IL); Itamar GAFNI, Tel-Aviv (IL); Shira COREM, Sdei Hemed (IL)

(73) Assignee: BETTERSEEDS LTD, Givat Chen (IL)

(21) Appl. No.: 18/271,624

(22) PCT Filed: Jan. 12, 2022
(86) PCT No.: PCT/IL2022/050049

§ 371 Date: Jul. 10, 2023

Related U.S. Application Data
(60) Provisional application No. 63/136,214, filed on Jan. 12, 2021.

(51) Int. Cl. A01H 6/54; A01H 5/10

(52) U.S. Cl. CPC A01H 6/542; A01H 5/10

(57) ABSTRACT

The present invention discloses a modified Soybean plant, plant part, or plant cell exhibiting reduced phytoestrogen content and/or increased protein content, wherein the modified Soybean plant, plant part, or plant cell comprises one or more gene editing derived mutations in a least one gene involved in phytoestrogen biosynthesis and/or protein accumulation. Methods for producing the same are further disclosed.

### FIELD OF THE INVENTION

The present disclosure concerns a method of modulating the expression of phytoestrogen synthesis genes and genes involved in protein accumulation thus generating Soybean plants with lower phytoestrogen levels and seeds with higher protein content.

### BACKGROUND OF THE INVENTION

Phytoestrogens are present in certain edible plants and being most abundant in Soybean (soy); they are structurally and functionally analogous to the estrogens. Phytoestrogens have been applied for compensation of hormone deficiency during menopause. At the same time, soy products are used in infant food and other foodstuffs. Furthermore, soy is applied as animal fodder, so that residual phytoestrogens and their active metabolites such as equol can remain in meat and influence the hormonal balance of the consumers. There have been reports on modified gender-related behavior or feminization in humans as consequence of soy consumption. In animals, the intake of phytoestrogens was reported to impact fertility, sexual development and behavior. Feminizing effects in humans can be subtle and identifiable only statistically in large populations.

Soybean is considered as a health food by some, with claims of taming hot flashes, warding off osteoporosis, and protecting against hormonal cancers like breast and prostate.

At the same time, soy is excluded by others for fear that it may cause breast cancer, thyroid problems, and dementia.

As a species within the legume family, nutrition scientists often label Soybean as a food with potential of significant health benefits. However, due to contrary research that suggests possible negative effects of soy in certain situations, there has been a hesitancy to undoubtedly promote soy.

Part of the uncertainty is due to the complexity of soy's effects on the body. Soybean is unique in that it contains a high concentration of isoflavones, a type of plant estrogen (phytoestrogen) that is similar in function to human estrogen but with much weaker effects. Soy isoflavones can bind to estrogen receptors in the body and cause either weak estrogenic or anti-estrogenic activity. The two major soy isoflavones are called genistein and daidzein. Soy isoflavones and soy protein appear to have different actions in the body. Coumestrol is a phytochemical found in Soybean and has been reported to possess α-glucosidase inhibitory and strong estrogen-like activities.

Aside from their phytoestrogen content, soy foods are rich in nutrients including B vitamins, fiber, potassium, magnesium, and high-quality protein. Unlike some plant proteins, Soybean protein is considered a complete protein, containing all nine essential amino acids that the body cannot make which must be obtained from the diet. Soy foods are also classified as fermented or unfermented. Fermented means that the soy food has been cultured with beneficial bacteria, yeast, or mold. Some believe that fermenting soy improves its digestibility and absorption in the body, as this process partially breaks down soy's sugar and protein molecules.

Soy contains a high concentration of essential amino acids and is the main source of protein in our global food supply. Soybean production has more than doubled over the last two decades. Almost 80% of the world's Soybean crop is fed to livestock, especially for beef, chicken, egg and dairy production (milk, cheeses, butter, yogurt, etc). Soy oil is used for cooking and can also be found in margarine, chocolate, ice cream or baked goods, as well as in cosmetics or soaps.

But the rising demand for Soybean has come at a cost. To produce Soybean, land is being converted from forests, savannahs and grasslands, endangering valuable habitats and species whilst putting at risk traditional, local livelihoods. Millions of hectares of important habitat like the Amazon rainforest, the Cerrado, the Atlantic Forest, the Gran Chaco and Chiquitano in South America, or the Northern Great Plains of the U.S. are being ploughed up to make room for more Soybean production. In addition, Soybean production generates greenhouse gases that contribute to climate change. Soybean is an intensively grown crop, with high demands for resources: particularly energy, water, agrochemicals and soil. Any change from natural vegetation or grazing lands to crops is likely to increase soil erosion and change the hydrological cycle.

In lieu of the above, it is a long felt and unmet need to provide a novel method of effectively and consistently eliminating or reducing phytoestrogen compounds in a Soybean plant to make the plant healthier for consumption of humans and in particular children and adolescents. In addition in lieu of above, it is highly important to increase the protein content in the Soybean plant to compensate for the dwindling natural resources that is associated with growing Soybean.

### SUMMARY OF THE INVENTION

It is therefore one object of the present invention to disclose a modified Soybean plant, plant part, or plant cell exhibiting reduced phytoestrogen content and/or increased protein content, wherein the modified Soybean plant, plant part, or plant cell comprises one or more gene editing derived mutations in at least one gene selected from Glyma.01G239600, Glyma.01G228700, Glyma.10G010500.1, Glyma.11 G010500, Glyma.13 G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

It is a further object of the present invention to disclose the modified Soybean plant, plant part, or plant cell as defined in any of the above, wherein the plant, plant part, or plant cell is characterized by reduced phytoestrogen content and/or increased protein content, as compared to a Soybean plant absent of said one or more mutations.

It is a further object of the present invention to disclose the modified Soybean plant, plant part, or plant cell as defined in any of the above, wherein the plant, plant part, or plant cell is characterized by modulated or altered expression of said at least one gene as compared to a Soybean plant absent of said one or more mutations.

It is a further object of the present invention to disclose the modified Soybean plant, plant part, or plant cell as defined in any of the above, wherein the plant, plant part, or plant cell is characterized by reduced expression of said at least one gene as compared to a Soybean plant absent of said one or more mutations.

It is a further object of the present invention to disclose the modified Soybean plant, plant part, or plant cell as defined in any of the above, wherein the plant, plant part, or plant cell comprises one or more gene editing derived mutations in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.11g011500, Glyma.02g005600, Glyma.17g030400 and Glyma19g030500 conferring reduced phytoestrogen content to said plant, plant part, or plant cell.

It is a further object of the present invention to disclose the modified Soybean plant, plant part, or plant cell as defined in any of the above, wherein the plant, plant part, or plant cell comprises one or more gene editing derived mutations in at least one gene selected from Glyma.17G036400, Glyma.17G036300 and Glyma.19g135000 conferring increased protein content in the Soybean plant, plant part, or plant cell.

It is a further object of the present invention to disclose the modified Soybean plant, plant part, or plant cell as defined in any of the above, wherein the Glyma.01G239600, Glyma.01G228700, Glyma.10G010500.1, Glyma.11 G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes comprise a genomic sequence as set forth in SEQ ID NO: 1, 220, 454, 674, 1014, 1305, 1491, 1798, 2090, 2200, 2296, 2630, 2810, 2973 and 3043, respectively, or a functional variant thereof.

It is a further object of the present invention to disclose the modified Soybean plant, plant part, or plant cell as defined in any of the above, wherein the functional variant has at least 80% sequence identity to said SEQ ID NO: 1, 220, 454, 674, 1014, 1305, 1491, 1798, 2090, 2200, 2296, 2630, 2810, 2973 and 3043.

It is a further object of the present invention to disclose the modified Soybean plant, plant part, or plant cell as defined in any of the above, wherein the sequence of a target editing region within the at least one gene is selected from the group comprising a) a nucleic acid sequence encoding the polypeptide comprising a sequence selected from the group comprising SEQ ID NO: 3, 222, 456, 676, 1016, 1307, 1493, 1800, 2092, 2202, 2298, 2632, 2812, 2975 and 3045, b) a nucleic acid sequence comprising a sequence selected from the group comprising SEQ ID NO: 1, 220, 454, 674, 1014, 1305, 1491, 1798, 2090, 2200, 2296, 2630, 2810, 2973 and 3043, and c) a nucleic acid sequence having at least 80% sequence identity to at least 200 contiguous nucleotides of a nucleic acid sequence selected from the group comprising SEQ ID NO: 1, 220, 454, 674, 1014, 1305, 1491, 1798, 2090, 2200, 2296, 2630, 2810, 2973 and 3043.

It is a further object of the present invention to disclose the modified Soybean plant, plant part, or plant cell as defined in any of the above, wherein the one or more mutations is introduced in planta using targeted genome modification.

It is a further object of the present invention to disclose the modified Soybean plant, plant part, or plant cell as defined in any of the above, wherein the one or more mutations is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

It is a further object of the present invention to disclose the modified Soybean plant, plant part, or plant cell as defined in any of the above, wherein said Cas gene is selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasY, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966 and any combination thereof.

It is a further object of the present invention to disclose the modified Soybean plant, plant part, or plant cell as defined in any of the above, wherein the at least one mutated gene is a CRISPR/Cas9-induced heritable mutated allele.

It is a further object of the present invention to disclose the modified Soybean plant, plant part, or plant cell as defined in any of the above, wherein the one or more mutations is selected from a silencing mutation, a knockdown mutation, a knockout mutation, a loss of function mutation, a downregulation mutation, an upregulation mutation or any combination thereof.

It is a further object of the present invention to disclose the modified Soybean plant, plant part, or plant cell as defined in any of the above, wherein the one or more mutations is in the coding region of said gene, a mutation in the regulatory region of said gene, or an epigenetic factor.

It is a further object of the present invention to disclose the modified Soybean plant, plant part, or plant cell as defined in any of the above, wherein said plant, plant part, or plant cell is homozygous for said at least one mutated gene.

It is a further object of the present invention to disclose the modified Soybean plant, plant part, or plant cell as defined in any of the above, wherein said plant, plant part, or plant cell is heterozygous for said at least one mutated gene.

It is a further object of the present invention to disclose the modified Soybean plant, plant part, or plant cell as defined in any of the above, wherein the one or more mutations is generated in planta via introduction of (a) Cas DNA and guide RNA (gRNA) sequence complementary to SEQ ID NO: 1, 220, 454, 674, 1014, 1305, 1491, 1798, 2090, 2200, 2296, 2630, 2810, 2973 and 3043, or (b) a ribonucleoprotein (RNP) complex comprising Cas protein and gRNA sequence complementary to SEQ ID NO: 1, 220, 454, 674, 1014, 1305, 1491, 1798, 2090, 2200, 2296, 2630, 2810, 2973 and 3043.

It is a further object of the present invention to disclose the modified Soybean plant, plant part, or plant cell as defined in any of the above, wherein said one or more mutations is generated in planta in said at least one Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes via introduction of (a) Cas DNA and guide RNA (gRNA) sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, respectively, and any combination thereof, or (b) a ribonucleoprotein (RNP) complex comprising Cas protein and gRNA sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, respectively, and any combination thereof.

It is a further object of the present invention to disclose the modified Soybean plant, plant part, or plant cell as defined in any of the above, wherein said gRNA sequence comprises a 3′ Protospacer Adjacent Motif (PAM) selected from NGG, NNGRRT, NNNNGATT, NNAGAAW and NAAAAC.

It is a further object of the present invention to disclose the modified Soybean plant, plant part, or plant cell as defined in any of the above, wherein the plant, plant part, or plant cell does not comprise a transgene.

According to a further embodiment of the present invention, the modified Soybean plant, plant part, or plant cell as defined in any of the above, wherein the plant part is a seed, tissue culture of regenerable cells, protoplasts, callus or pollen.

It is a further object of the present invention to disclose the modified Soybean plant, plant part, or plant cell as defined in any of the above, wherein the plant, plant part, or plant cell has a content of phytoestrogen that is reduced by about 10%, about 20%, about 30%, about 50%, about 60%, about 70%, about 80%, or about 90% compared to the content of phytoestrogen in a Soybean plant, plant part, or plant cell absent of said one or more mutations.

It is a further object of the present invention to disclose the modified Soybean plant, plant part, or plant cell as defined in any of the above, wherein the phytoestrogen is selected from isoflavone and/or Coumestrol.

It is a further object of the present invention to disclose the modified Soybean plant, plant part, or plant cell as defined in any of the above, wherein the plant, plant part, or plant cell has a protein content that is elevated by about 10%, about 20%, about 30%, about 50%, about 60%, about 70%, about 80%, or about 90% compared to the protein content in a Soybean plant, plant part, or plant cell absent of said one or more mutations.

It is a further object of the present invention to disclose Soybean seed, pollen, plant parts or progeny of the modified Soybean plant as defined in any of the above, wherein said seed, pollen, plant parts or progeny comprises said one or more gene editing derived mutations in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11 g011500, Glyma.02g005600, Glyma.17g030400 and Glyma19g030500.

It is a further object of the present invention to disclose food and non-food products incorporating the modified Soybean plant, plant part or plant cell as defined in any of the above.

It is a further object of the present invention to disclose food and non-food products incorporating a seed of a Soybean plant as defined in any of the above.

It is a further object of the present invention to disclose a method for producing a modified Soybean plant, plant part, or plant cell exhibiting reduced phytoestrogen content and/or increased protein content using targeted genome modification, comprising steps of: (a) identifying a gene involved in phytoestrogen synthesis and/or increased protein content of a Soybean plant species; (b) synthesizing or designing a gRNA expression cassette corresponding to a target editing region along the Soybean genome, within the identified gene locus; (c) transforming said Soybean plant, plant part, or plant cell with CRISPR/Cas9 system; (d) screening the genome of said transformed plant, plant part, or plant cell for induced targeted mutation, e.g. loss of function mutation in the identified gene involved in phytoestrogen synthesis and/or increased protein content; (e) selecting said Soybean plant, plant part, or plant cell expressing desired one or more mutations in the editing target region, said mutations confer reduced phytoestrogen content and/or increased protein content; and (f) optionally, regenerating a plant from said transformed plant, plant part, or plant cell plant cell nucleus, or plant tissue and optionally screening said regenerated plants for a Soybean plant, with decreased phytoestrogen content and/or increased protein content.

It is a further object of the present invention to disclose the method as defined in any of the above, wherein the gRNA and its corresponding PAM is complementary to a sequence of a gene selected from a group comprising at least one of Glyma.01G239600, Glyma.01G228700, Glyma.10G010500.1, Glyma.11 G010500, Glyma.13G173500, Glyma.14G005700, Glyma.17G064400, Glyma.16G149300, Glyma.17G036400, Glyma.17G036300, Glyma.19g135000, Glyma.11 g011500, Glyma.02g005600, Glyma.17g030400 and Glyma19g030500.

It is a further object of the present invention to disclose a method for producing a modified Soybean plant, plant part, or plant cell exhibiting reduced phytoestrogen content and/or increased protein content using targeted genome modification comprising introducing into the genome of the plant, plant part, or plant cell one or more mutations in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

It is a further object of the present invention to disclose the method as defined in any of the above, wherein the plant, plant part, or plant cell is characterized by reduced phytoestrogen content and/or increased protein content, as compared to a Soybean plant absent of said one or more mutations.

It is a further object of the present invention to disclose the method as defined in any of the above, wherein the plant, plant part, or plant cell is characterized by modulated or altered expression of said at least one gene as compared to a Soybean plant absent of said one or more mutations.

It is a further object of the present invention to disclose the method as defined in any of the above, wherein the plant, plant part, or plant cell is characterized by reduced expression of said at least one gene as compared to a Soybean plant absent of said one or more mutations.

It is a further object of the present invention to disclose the method as defined in any of the above, wherein the plant, plant part, or plant cell comprises one or more gene editing derived mutations in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.11g011500, Glyma.02g005600, Glyma.17g030400 and Glyma19g030500 conferring reduced phytoestrogen content to said plant, plant part, or plant cell.

It is a further object of the present invention to disclose the method as defined in any of the above, wherein the plant, plant part, or plant cell comprises one or more gene editing derived mutations in at least one gene selected from Glyma.17G036400, Glyma.17G036300 and Glyma.19g135000 conferring increased protein content in the Soybean plant, plant part, or plant cell.

It is a further object of the present invention to disclose the method as defined in any of the above, wherein the Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes comprise a genomic sequence as set forth in SEQ ID NO: 1, 220, 454, 674, 1014, 1305, 1491, 1798, 2090, 2200, 2296, 2630, 2810, 2973 and 3043, respectively, or a functional variant thereof.

It is a further object of the present invention to disclose the method as defined in any of the above, wherein the functional variant has at least 80% sequence identity to said SEQ ID NO: 1, 220, 454, 674, 1014, 1305, 1491, 1798, 2090, 2200, 2296, 2630, 2810, 2973 and 3043.

It is a further object of the present invention to disclose the method as defined in any of the above, wherein the sequence of a target editing region within the at least one gene is selected from the group comprising a) a nucleic acid sequence encoding the polypeptide comprising a sequence selected from the group comprising SEQ ID NO: 3, 222, 456, 676, 1016, 1307, 1493, 1800, 2092, 2202, 2298, 2632, 2812, 2975 and 3045, b) a nucleic acid sequence comprising a sequence selected from the group comprising SEQ ID NO: 1, 220, 454, 674, 1014, 1305, 1491, 1798, 2090, 2200, 2296, 2630, 2810, 2973 and 3043, and c) a nucleic acid sequence having at least 80% sequence identity to at least 200 contiguous nucleotides of a nucleic acid sequence selected from the group comprising SEQ ID NO: 1, 220, 454, 674, 1014, 1305, 1491, 1798, 2090, 2200, 2296, 2630, 2810, 2973 and 3043.

It is a further object of the present invention to disclose the method as defined in any of the above, wherein the one or more mutations is introduced in planta using targeted genome modification.

It is a further object of the present invention to disclose the method as defined in any of the above, wherein the one or more mutations is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

It is a further object of the present invention to disclose the method as defined in any of the above, wherein said Cas gene is selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasY, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966 and any combination thereof.

It is a further object of the present invention to disclose the method as defined in any of the above, wherein the at least one mutated gene is a CRISPR/Cas9-induced heritable mutated allele.

It is a further object of the present invention to disclose the method as defined in any of the above, wherein the one or more mutations is selected from a silencing mutation, a knockdown mutation, a knockout mutation, a loss of function mutation, a downregulation mutation, an upregulation mutation or any combination thereof.

It is a further object of the present invention to disclose the method as defined in any of the above, wherein the one or more mutations is in the coding region of said gene, a mutation in the regulatory region of said gene, or an epigenetic factor.

It is a further object of the present invention to disclose the method as defined in any of the above, wherein said plant, plant part, or plant cell is homozygous for said at least one mutated gene.

It is a further object of the present invention to disclose the method as defined in any of the above, wherein said plant, plant part, or plant cell is heterozygous for said at least one mutated gene.

It is a further object of the present invention to disclose the method as defined in any of the above, wherein the one or more mutations is generated in planta via introduction of (a) Cas DNA and guide RNA (gRNA) sequence complementary to SEQ ID NO: 1, 220, 454, 674, 1014, 1305, 1491, 1798, 2090, 2200, 2296, 2630, 2810, 2973 and 3043, or (b) a ribonucleoprotein (RNP) complex comprising Cas protein and gRNA sequence complementary to SEQ ID NO: 1, 220, 454, 674, 1014, 1305, 1491, 1798, 2090, 2200, 2296, 2630, 2810, 2973 and 3043.

It is a further object of the present invention to disclose the method as defined in any of the above, wherein said one or more mutations is generated in planta in said at least one Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes via introduction of (a) Cas DNA and guide RNA (gRNA) sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, respectively, or (b) a ribonucleoprotein (RNP) complex comprising Cas protein and gRNA sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, respectively.

It is a further object of the present invention to disclose the method as defined in any of the above, wherein said gRNA sequence comprises a 3′ Protospacer Adjacent Motif (PAM) selected from NGG, NNGRRT, NNNNGATT, NNAGAAW and NAAAAC.

It is a further object of the present invention to disclose the method as defined in any of the above, wherein the transformation is carried out to deliver an expression cassette comprising a) a selection marker, b) a nucleotide sequence encoding one or more gRNA molecules comprising a sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, and c) a nucleotide sequence encoding a Cas molecule.

It is a further object of the present invention to disclose the method as defined in any of the above, wherein the method comprises administering to the Soybean plant, plant part, or plant cell a nucleic acid composition comprising: a) a first nucleotide sequence encoding a gRNA molecule, and b) a second nucleotide sequence encoding the Cas molecule.

It is a further object of the present invention to disclose the method as defined in any of the above, wherein the CRISPR/Cas system is delivered to the cell by a plant virus.

It is a further object of the present invention to disclose the method as defined in any of the above, wherein the method comprises steps of (a) introducing into a Soybean plant or plant part or cell thereof (i) at least one RNA-guided endonuclease comprising at least one nuclear localization signal or nucleic acid encoding at least one RNA-guided endonuclease comprising at least one nuclear localization signal, (ii) at least one guide RNA (gRNA) or DNA encoding at least one gRNA, and, optionally, (iii) at least one donor polynucleotide; and (b) culturing the Soybean plant or plant part or cell thereof such that each gRNA directs an RNA-guided endonuclease to a target site in the chromosomal sequence where the RNA-guided endonuclease introduces a double-stranded break in the target site, and the double-stranded break is repaired by a DNA repair process such that the chromosomal sequence is modified, wherein the target site is located in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 and the chromosomal modification interrupts or interferes with transcription and/or translation of the at least one gene.

It is a further object of the present invention to disclose the method as defined in any of the above, wherein the RNA-guided endonuclease is derived from a clustered regularly interspersed short palindromic repeats (CRISPR)/CRISPR-associated (Cas) system.

It is a further object of the present invention to disclose the method as defined in any of the above, wherein introduction of a genome modification does not insert exogenous genetic material, and produces a non-naturally occurring or modified Soybean plant, plant part or cell thereof.

It is a further object of the present invention to disclose the method as defined in any of the above, further comprises steps of (a) identifying at least one locus within a DNA sequence of a Soybean plant or a cell thereof selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes; (b) identifying at least one custom endonuclease recognition sequence within the at least one locus of the gene; and (c) identifying the Soybean plant, a cell thereof, or a progeny cell thereof as comprising a modification in the loci of the at least one gene, said modification confers modulated or altered expression, e.g. silencing, of at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

It is a further object of the present invention to disclose the method as defined in any of the above, wherein the plant, plant part, or plant cell does not comprise a transgene.

It is a further object of the present invention to disclose the method as defined in any of the above, wherein the plant part is a seed, tissue culture of regenerable cells, protoplasts, callus or pollen.

It is a further object of the present invention to disclose the method as defined in any of the above, wherein the plant, plant part, or plant cell has a content of phytoestrogen that is reduced by about 10%, about 20%, about 30%, about 50%, about 60%, about 70%, about 80%, or about 90% compared to the content of phytoestrogen in a Soybean plant, plant part, or plant cell absent of said one or more mutations.

It is a further object of the present invention to disclose the method as defined in any of the above, wherein the phytoestrogen is selected from isoflavone and/or Coumestrol.

It is a further object of the present invention to disclose the method as defined in any of the above, wherein the plant, plant part, or plant cell has a protein content that is elevated by about 10%, about 20%, about 30%, about 50%, about 60%, about 70%, about 80%, or about 90% compared to the protein content in a Soybean plant, plant part, or plant cell absent of said one or more mutations.

It is a further object of the present invention to disclose the method as defined in any of the above, wherein the method further comprises steps of analyzing the Soybean plants or progeny thereof, plant part, or plant cell by a) isolating genomic DNA from the mutated plant material or its progeny; and b) amplifying segments of a gene involved in phytoestrogen synthesis and/or a gene associated with protein accumulation such as fatty acid transport or sugar metabolism, in the isolated genomic DNA, using primers specific to at least one of Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes or to the DNA sequences adjacent to the at least one gene.

It is a further object of the present invention to disclose a modified Soybean seed, pollen, plant parts or progeny with reduced phytoestrogen content and/or increased protein content, produced by the method as defined in any of the above, wherein said seed, pollen, plant parts or progeny comprises said one or more gene editing derived mutations in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

It is a further object of the present invention to disclose a method for reducing phytoestrogen content and/or increasing protein content in a Soybean plant, plant part or plant cell, using targeted genome modification, comprising introducing into the genome of the plant, plant part, or plant cell one or more mutations in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

It is a further object of the present invention to disclose a method for increasing Soybean yield using targeted genome modification, comprising introducing into the genome of the Soybean plant, plant part, or plant cell one or more mutations in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

It is a further object of the present invention to disclose the method as defined in any of the above, wherein the plant part is selected from the group consisting of seeds, pollen, plant cells, or plant tissue.

It is a further object of the present invention to disclose the method as defined in any of the above, wherein the plant, plant part, or plant cell comprises one or more gene editing derived mutations in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.11g011500, Glyma.02g005600, Glyma.17g030400 and Glyma19g030500 conferring reduced phytoestrogen content to said plant, plant part, or plant cell.

It is a further object of the present invention to disclose the method as defined in any of the above, wherein the plant, plant part, or plant cell comprises one or more gene editing derived mutations in at least one gene selected from Glyma.17G036400, Glyma.17G036300 and Glyma.19g135000 conferring increased protein content in the Soybean plant, plant part, or plant cell.

It is a further object of the present invention to disclose the method as defined in any of the above, wherein the Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes comprise a genomic sequence as set forth in SEQ ID NO: 1, 220, 454, 674, 1014, 1305, 1491, 1798, 2090, 2200, 2296, 2630, 2810, 2973 and 3043, respectively, or a functional variant thereof.

It is a further object of the present invention to disclose the method as defined in any of the above, wherein the functional variant has at least 80% sequence identity to said SEQ ID NO: 1, 220, 454, 674, 1014, 1305, 1491, 1798, 2090, 2200, 2296, 2630, 2810, 2973 and 3043.

It is a further object of the present invention to disclose the method as defined in any of the above, wherein said one or more mutations is generated in planta in said at least one Glyma.01G239600, Glyma.01G228700, Glyma.10G010500.1, Glyma.11 G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes via introduction of (a) Cas DNA and guide RNA (gRNA) sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, respectively, or (b) a ribonucleoprotein (RNP) complex comprising Cas protein and gRNA sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, respectively.

It is a further object of the present invention to disclose a construct comprising or encoding (a) a nucleotide sequence encoding a Cas molecule and one or more gRNA molecules comprising a sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042, SEQ ID NO: 3046-3268, and any combination thereof, or (b) a ribonucleoprotein (RNP) complex comprising Cas protein and one or more gRNA molecules comprising a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, and any combination thereof.

It is a further object of the present invention to disclose a method of modifying the phytoestrogen content and/or protein content of a Soybean plant, plant part, or plant cell, comprising introducing into a Soybean plant, plant part, or plant cell the construct as defined above.

It is a further object of the present invention to disclose an isolated polynucleotide sequence comprising at least 80% sequence identity to a polynucleotide sequence selected from the group consisting of SEQ ID NO: 1, 2, 220, 221, 454, 455, 674, 675, 1014, 1015, 1305, 1306, 1491, 1492, 1798, 1799, 2090, 2091, 2200, 2201, 2296, 2297, 2630, 2631, 2810, 2811, 2973, 2974, 3043 and 3044.

It is a further object of the present invention to disclose an isolated polypeptide sequence comprising at least 80% sequence identity to SEQ ID NO: 3, 222, 456, 676, 1016, 1307, 1493, 1800, 2092, 2202, 2298, 2632, 2812, 2975 and 3045.

It is a further object of the present invention to disclose a use of a nucleic acid sequence comprising at least 80% sequence identity to a sequence as set forth in at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2633-2809, SEQ ID NO: 2813-2972, SEQ ID NO: 2976-3042 or SEQ ID NO: 3046-3268 and any combination thereof for targeted gene editing of at least one Soybean gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.11g011500, Glyma.02g005600, Glyma.17g030400 and Glyma19g030500, respectively, said gene editing confers reduced phytoestrogen content in a Soybean plant, plant part or plant cell.

It is a further object of the present invention to disclose a use of a nucleic acid sequence comprising at least 80% sequence identity to a sequence as set forth in at least one of SEQ ID NO: 2093-2199, SEQ ID NO: 2203-2295, SEQ ID NO: 2299-2629 and any combination thereof, for targeted gene editing of at least one Soybean gene selected from Glyma.17G036400, Glyma.17G036300, Glyma.19g135000, respectively, said gene editing confers increased protein content in a Soybean plant, plant part or plant cell.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

The present disclosure relates to conferring desirable traits in Soybean plants. More particularly, the current invention pertains to producing Soybean plants with improved protein content and containing less xenoestrogen, by manipulating genes controlling fatty acid transport and/or sugar metabolism, and phenolic compounds, respectively.

The term “Xenoestrogen” as used herein refers to a type of xenohormone that imitates estrogen. Xenoestrogen can be either synthetic or natural chemical compound. Synthetic xenoestrogens include some widely used industrial compounds, such as PCBs, BPA, and phthalates, which have estrogenic effects on a living organism even though they differ chemically from the estrogenic substances produced internally by the endocrine system of any organism. Natural xenoestrogens include phytoestrogens which are plant-derived xenoestrogens. Because the primary route of exposure to these compounds is by consumption of phytoestrogenic plants, they are also called “dietary estrogens”. Mycoestrogens, estrogenic substances from fungi, are another type of xenoestrogen that are also considered mycotoxins.

Xenoestrogens are clinically significant because they can mimic the effects of endogenous estrogen and thus have been implicated in disorders of the reproductive system.

A phytoestrogen is a plant-derived xenoestrogen not generated within the endocrine system, but consumed by eating phytoestrogenic plants. It is a diverse group of naturally occurring nonsteroidal plant compounds that, because of its structural similarity with estradiol (1743-estradiol), have the ability to cause estrogenic and/or antiestrogenic effects.

The similarities, at molecular level, of an estrogen and a phytoestrogen allow them to mildly mimic and sometimes act as an antagonist of estrogen.

It is also within the scope of the current invention that phytoestrogens are naturally occurring, polyphenolic, non-steroidal plant compounds that are structurally similar to 170-estradiol and have estrogenic and/or antiestrogenic effects. These effects are mediated by binding to estrogen receptors, by alterations in the concentrations of endogenous estrogens, and by binding to or stimulation of the synthesis of sex hormone binding globulin.

There are four main groups of phytoestrogens: isoflavonoids, flavonoids, stilbenes, and lignans. Of these, the most commonly occurring are the flavonoids (of which the coumestans, prenylated flavonoids, and isoflavones have the greatest estrogenic effects) and the lignans. The isoflavonoids include genistein, daidzein, coumestrol, and equol; the prenylated flavonoids include 8-prenylnaringenin, the stilbenes include resveratrol, and the lignans include enterodiol and enterolactone. Mycoestrogens (mycotoxins) are metabolites of Fusarium, a fungus that is often found in pastures and in alfalfa and clover.

It is herein acknowledged that a wide range of plants and the products derived from them, such as soya milk and tofu, contain estrogens (isoflavones or lignans) with some chemical affinity for estradiol; the concentrations of isoflavone are particularly high in the Soybean plant.

According to further aspects of the present invention, isoflavones are a class of flavonoids, and the main isoflavones of soybeans are daidzin, genistin, and glycitin.

The terms “Soybean” or “Soya” or “Soy” or “Soya bean” or Soy bean” refers to the plant species Glycine max of the genus Glycine (family Fabaceae), which is a species of legume native to East Asia. Traditional unfermented food uses of Soybeans include soy milk, from which tofu and tofu skin are made. Fermented soy foods include soy sauce, fermented bean paste, natto, and tempeh. Fat-free (defatted) soybean meal is a significant and cheap source of protein for animal feeds and many packaged meals. For example, Soybean products, such as textured vegetable protein (TVP), are ingredients in many meat and dairy substitutes.

As used herein, the term “phytoestrogen synthesis gene” or “phytoestrogen biosynthesis gene” refers to any gene involved in isoflavone/coumestrol (CM)/phytoestrogen biosynthesis in Soybean. In the context of the present invention it refers to a gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.11g011500, Glyma.02g005600, Glyma.17g030400 and Glyma19g030500, homologs, orthologs, paralogs and variants thereof.

As used herein, the term “protein content” or “protein accumulation” associated gene refers to any gene involved in or associated with protein accumulation or increasing protein content in Soybean. In the context of the present invention it refers to a gene selected from Glyma.17G036400, Glyma.17G036300 and Glyma.19g135000, homologs, orthologs, paralogs and variants thereof.

As used herein, the term “modified” or “non-naturally occurring” when used in reference to a Soybean plant means a Soybean plant that has been genetically modified by human, for example, by induction of a mutation in a gene or regulatory element of the gene by for example gene editing, such as by the CRISPR/Cas system targeted by specific guide RNA (gRNA) molecules. Furthermore, a plant generated by cross breeding different strains and varieties is also considered a “non-naturally occurring plant” because the selection and breeding is performed by human intervention. In contrast, a Soybean plant containing only spontaneous or naturally occurring mutations is not a “modified” or “non-naturally occurring Soybean plant” as defined herein. One skilled in the art understands that while a modified or non-naturally occurring Soybean plant typically has a nucleotide sequence that is altered as compared to a similar unmodified or naturally occurring Soybean plant, a modified or non-naturally occurring Soybean plant also can be genetically modified by human intervention without altering its nucleotide sequence, for example, by modifying its methylation pattern.

According to one embodiment, the present invention provides a modified Soybean plant, plant part, or plant cell exhibiting reduced phytoestrogen content and/or increased protein content, wherein the modified Soybean plant, plant part, or plant cell comprises one or more gene editing derived mutations in at least one gene selected from Glyma.01G239600, Glyma.01G228700, Glyma.10G010500.1, Glyma.11G010500, Glyma.13 G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

According to a further embodiment of the present invention, the plant, plant part, or plant cell is characterized by reduced phytoestrogen content and/or increased protein content, as compared to a Soybean plant absent of said one or more mutations.

According to a further embodiment of the present invention, the plant, plant part, or plant cell is characterized by modulated or altered expression of said at least one gene as compared to a Soybean plant absent of said one or more mutations.

According to a further embodiment of the present invention, the plant, plant part, or plant cell is characterized by reduced expression of said at least one gene as compared to a Soybean plant absent of said one or more mutations.

According to a further embodiment of the present invention, the plant, plant part, or plant cell comprises one or more gene editing derived mutations in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.11g011500, Glyma.02g005600, Glyma.17g030400 and Glyma19g030500 conferring reduced phytoestrogen content to said plant, plant part, or plant cell.

According to a further embodiment of the present invention, the plant, plant part, or plant cell comprises one or more gene editing derived mutations in at least one gene selected from Glyma.17G036400, Glyma.17G036300 and Glyma.19g135000 conferring increased protein content in the Soybean plant, plant part, or plant cell.

The present invention further provides a method for producing a modified Soybean plant, plant part, or plant cell exhibiting reduced phytoestrogen content and/or increased protein content using targeted genome modification comprising steps of: (a) identifying a gene involved in phytoestrogen synthesis and/or increased protein content of a Soybean plant species; (b) synthesizing or designing a gRNA expression cassette corresponding to a target editing region along the Soybean genome, within the identified gene locus; (c) transforming said Soybean plant, plant part, or plant cell with CRISPR/Cas9 system; (d) screening the genome of said transformed plant, plant part, or plant cell for induced targeted mutation, e.g. loss of function mutation in the identified gene involved in phytoestrogen synthesis and/or increased protein content; (e) selecting said Soybean plant, plant part, or plant cell expressing desired one or more mutations in the editing target region, said mutations confer reduced phytoestrogen content and/or increased protein content; and (f) optionally, regenerating a plant from said transformed plant, plant part, or plant cell plant cell nucleus, or plant tissue and optionally screening said regenerated plants for a Soybean plant, with decreased phytoestrogen content and/or increased protein content.

It is further within the scope of the present invention that the gRNA and its corresponding PAM is complementary to a sequence of a gene selected from a group comprising at least one of Glyma.01G239600, Glyma.01G228700, Glyma.10G010500.1, Glyma.11 G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

According to a further embodiment, the present invention provides a method for producing a modified Soybean plant, plant part, or plant cell exhibiting reduced phytoestrogen content and/or increased protein content using targeted genome modification comprising introducing into the genome of the plant, plant part, or plant cell one or more mutations in at least one gene selected from Glyma.01G239600, Glyma.01 G228700, Glyma.10G010500.1, Glyma.11 G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

The method of the present invention is based on gene editing of the Soybean plant genome at specific nucleic acid sequences, which result in a set of desired traits such as decreased phytoestrogens (e.g. phytoestrogen free) and higher protein content or protein accumulation.

The challenge here is to efficiently induce precise and predictable targeted mutations pivotal to reducing phytoestrogen levels and or increasing protein content in the Soybean plant using the CRISPR/Cas9 system.

A significant added value of gene editing is that it does not qualify as transgene modification so the resultant transgene-free plant will not be considered a GMO plant/product in many significant territories. While the exact and operational definition of genetically modified is hotly debated and contested, it is generally agreed upon and accepted that genetic modification refers to plants and animals that have been altered in a way that wouldn't have arisen naturally through evolution. The clearest and most obvious example is a transgenic organism whose genome now incorporates a gene from another species, generating a novel trait to that organism, such as pest resistance. The situation is different with genome editing, as the CRISPR machinery is not necessarily integrated into the plant genome, it is used transiently to create the desired mutation and only the editing event is inherited to the next generation.

The present invention disclosed herein provides a method for producing a plant with decreased phytoestrogen compounds (e.g. isoflavones, Coumestrols) and/or higher content of protein when compared to a corresponding wild type, or non-edited Soybean plant. The present invention provides plant, plants cell, plant parts or their derivatives exhibiting decreased levels of phytoestrogen and higher protein content achieved by gene-editing and plants comprised of such cells, progeny, seed and pollen derived from such plants, and methods of making and methods of using such plant cell(s) or plant(s), progeny, seed(s), pollen or any other plant part thereof. Particularly, said improved trait(s) are manifested by reducing phytoestrogen levels and/or increasing seed protein content in the Soybean plant.

Gene Editing

Reference is now made to gene editing technique used in the present invention to produce Soybean plants with improved traits (e.g. reduced phytoestrogen levels and/or higher protein content).

Without wishing to be bound by theory, mutation breeding refers to a host of techniques designed to rapidly and effectively induce desired or remove unwanted traits via artificial mutations in a target organism. Gene editing is such a mutation breeding tool which offers significant advantages over traditional genetic modification.

Genetic transformation (i.e. traditional genetic modification) is a molecular technology involving inserting a DNA sequence of interest, coding for a desirable trait, into an organism's genome. Gene editing is a mutation breeding tool which allows precise, targeted modification of the genome. It achieves a result when a DNA nuclease (a protein complex from the Cas family) is precisely directed toward an exact genome locus using a guide RNA, and then cleaves the genome at that site.

One advantage of using the CRISPR/Cas system over traditional genetic modification is that the Cas protein family are easily programmed to make a DNA double strand break (DSB) at any desired loci. The initial cleavage is followed by repairing chromosomal DSBs. There are two major cellular repair pathways in that respect: Non-homologous end joining (NHEJ) and Homology directed repair (HDR).

In some aspects, this invention concerns NHEJ pathway which is active throughout the cell cycle and has a higher capacity for repair, as there is no requirement for a repair template (sister chromatid or homologue) or extensive DNA synthesis. NHEJ also finishes repair of most types of breaks in tens of minutes—an order of magnitude faster than HDR. NHEJ-mediated repair of DSBs is useful if the intent is to make a null allele (knockout) in a gene of interest, as it is prone to generating indel errors. Indel errors generated in the course of repair by NHEJ are typically small (1-10 bp) but extremely heterogeneous. There is consequently a relatively high chance of causing a frameshift mutation. Of some importance, the deletion can be less heterogeneous when constrained by sequence identities in flanking sequence (microhomologies).

Additionally, there is no foreign DNA left over in the plant after selection for plants which contain the desired editing event and do not carry the CRISPR/Cas machinery. This significant advantage has allowed gene editing to be viewed by many legal systems around the world as GMO-free.

Significant advances have been made recently in an attempt to more efficiently target and cleave genomic DNA by site specific nucleases [e.g. zinc finger nucleases (ZFNs), meganucleases, transcription activator-like effector nucleases (TALENS)]. More recently, RNA-guided endonucleases (RGENs) have been introduced, and they are directed to their target sites by a complementary RNA molecule. These systems have a DNA-binding domain that localizes the nuclease to a target site. The site is then cut by the nuclease. It is herein acknowledged that these systems are used to induce targeted mutagenesis, induce targeted deletions of cellular DNA sequences, and facilitate targeted recombination of an exogenous donor DNA polynucleotide within a predetermined genomic locus. Most notable and successful of RGENs is Clustered Regularly Interspaced Short Palindromic Repeats/CRISPR-associated nuclease (CRISPR/Cas) with an engineered crRNA/tracr RNA. CRISPR/Cas9 are cognates that find each other on the target DNA.

It is herein further acknowledged that the CRISPR-Cas9 system has rapidly become a tool of choice in gene editing because it is faster, cheaper, more accurate, and more efficient than other available RGENs. A small fragment of RNA with a short “guide” sequence (gRNA) that binds to a specific target sequence of DNA in a genome, is created. The RNA also binds to the Cas9 enzyme. As in bacteria, the modified RNA is used to recognize the DNA sequence, and the Cas9 enzyme cuts the DNA at the targeted location. Although Cas9 is the enzyme that is used most often, other enzymes (for example Cpf1) can also be used. Once the DNA is cut, the cell's own DNA repair machinery add or delete pieces of genetic material resulting in mutation.

Ribonucleoprotein Protein Complex (RNP)

Ribonucleoprotein protein complex is formed when a Cas protein is incubated with gRNA molecules and then transformed into cells in order to induce editing events in the cell. RNP's can be delivered using biolistics.

Biolistics

Biolistics is a method for the delivery of nucleic acid and or proteins to cells by high-speed particle bombardment. The technique uses a pressurized gun (gene gun) to forcibly propel a payload comprised of an elemental particle of a heavy metal coated with plasmid DNA to transform plant cellular organelles. After the DNA-carrying vector has been delivered, the DNA is used as a template for transcription and sometimes it integrates into a plant chromosome (“stable” transformation). If the vector also delivered a selectable marker, then stably transformed cells can be selected and cultured. Transformed plants can become totipotent and even display novel and heritable phenotypes.

It is noted that the skeletal biolistic vector design includes not only the desired gene to be inserted into the cell, but also promoter and terminator sequences as well as a reporter gene used to enable the ensuing detection and removal cells which failed to incorporate the exogenous DNA.

According to further aspects of the present invention, in addition to DNA, the use of a Cas9 protein and a gRNA molecule could be used for biolistic delivery. The advantage of using a protein and a RNA molecule is that the complex initiates editing upon reaching the cell nucleus: when using DNA for editing, the DNA first has to be transcribed in the nucleus but when using RNA for editing, RNA is translated already in the cytoplasm. This forces the Cas protein to shuttle back to the nucleus, find the relevant guides and only then can editing be achieved.

As used herein, the term “CRISPR” refers to an acronym that means Clustered Regularly Interspaced Short Palindromic Repeats of DNA sequences. CRISPR is a series of repeated DNA sequences with unique DNA sequences in between the repeats. RNA transcribed from the unique strands of DNA serves as guides for directing cleaving. CRISPR is used as a gene editing tool. In one embodiment, CRISPR is used in conjunction with (but not limited to) Cpf1, Cas9, Cas12, Cas13, Cas14, CasX or CasY.

As used herein, the term “transformation” refers to the deliberate insertion of genetic material into plant cells. In one embodiment transformation is executed using, but not limited to, bacteria and/or virus. In another embodiment, transformation is executed via biolistics using, but not limited to, DNA or RNPs.

Thus construct introduction into the plant cells may be performed via Agrobacterium infiltration, virus based plasmids for delivery of the genome editing molecules or mechanical insertion such as polyethylene glycol (PEG) mediated DNA transformation, electroporation or gene gun biolistics.

It is further within the scope of the present invention that the step of screening the genome of said transformed plant cells for induced targeted (e.g. loss of function) mutation comprises steps of obtaining a nucleic acid sample of said transformed plant and performing a nucleic acid amplification and optionally restriction enzyme digestion to detect a mutation in said at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11 g011500, Glyma.02g005600, Glyma.17g030400 and Glyma19g030500.

As used herein, the term “Cas” refers to CRISPR associated proteins that act as enzymes cutting the genome at specific sequences. Cas9 refers to a specific group of proteins known in the art. RNA molecules direct various classes of Cas enzymes to cut a certain sequence found in the genome. In one embodiment, the CRISPR/Cas9 system cleaves one or two chromosomal strands at known DNA sequence. In one embodiment, one of the two chromosomal strands is mutated. In one embodiment, two of the two chromosomal strands are mutated.

As used herein, the term “chromosomal strand” refers to a sequence of DNA within the chromosome. When the CRISPR/Cas9 system cleaves the chromosomal strands, the strands are cut leaving the possibility of one or two strands being mutated, either the template strand or coding strand.

As used herein, the term “PAM” (protospacer adjacent motif) refers to a targeting component of the transformation expression cassette which is a very short (2-6 base pair) DNA sequence immediately following the DNA sequence targeted by the Cas9 nuclease in the CRISPR system.

Within the context of this disclosure, other examples of endonuclease enzymes include, but are not limited to, Cpf1, Cas9, Cas12, Cas13, Cas14, CasX or CasY.

It is within the scope that the current invention concerns or encompass any of the phytoestrogen biosynthesis and elevated protein content associated genes in the Soybean plant, and not limited only to the genes listed in Table 1.

TABLE 1
Soybean genes associate with phytoestrogen biosynthesis and protein accumulation

| | | Genomic | Coding | | |
|---|---|---|---|---|---|
| | | polynucleotide | polynucleotide | Polypeptide | gRNA |
| | | sequence | sequence (CDS) | sequence | sequence |
| Gene annotation | Gene name | SEQ ID NO | SEQ ID NO | SEQ ID NO | SEQ ID NO |
| Glyma.01G239600 | 2- | 1 | 2 | 3 | 4-219 |
| | hydroxyisofavanone | | | | |
| | dehydratase, | | | | |
| | HIDH | | | | |
| Glyma.01G228700 | chalcone synthase, | 220 | 221 | 222 | 223-453 |
| | CHS7 | | | | |
| Glyma.10G010500.1 | Pectinesterase | 454 | 455 | 456 | 457-673 |
| Glyma.11G010500 | 4-coumarate:CoA | 674 | 675 | 676 | 677-1013 |
| | ligase, 4CL13 | | | | |
| Glyma.13G173500 | isofavone synthase, | 1014 | 1015 | 1016 | 1017-1304 |
| | IFS2 | | | | |
| Glyma.14G005700 | chalcone reductase, | 1305 | 1306 | 1307 | 1308-1490 |
| | CHR14 | | | | |
| Glyma.17G064400 | 4-coumarate:CoA | 1491 | 1492 | 1493 | 1494-1797 |
| | ligase | | | | |
| Glyma.16G149300 | cytochrome P450 | 1798 | 1799 | 1800 | 1801-2089 |
| | hydroxylase | | | | |
| Glyma.17G036400 | GmC/VIF2 | 2090 | 2091 | 2092 | 2093-2199 |
| Glyma.17G036300 | GmCIF1 | 2200 | 2201 | 2202 | 2203-2295 |
| Glyma.19g135000 | Fatty acid export | 2296 | 2297 | 2298 | 2299-2629 |
| Glyma.11g011500 | Chalcone synthase | 2630 | 2631 | 2632 | 2633-2809 |
| | 8 | | | | |
| Glyma.02g005600 | Transcription factor | 2810 | 2811 | 2812 | 2813-2972 |
| | MYB13 | | | | |
| Glyma.17g030400 | MLP-like protein | 2973 | 2974 | 2975 | 2976-3042 |
| | 423 | | | | |
| Glyma19g030500 | HXXXD-type acyl- | 3043 | 3044 | 3045 | 3046-3268 |
| | transferase family | | | | |
| | protein | | | | |
| | malonyl- | | | | |
| | CoA:isoflavone 7- | | | | |
| | O-glucoside-6″-O- | | | | |
| | malonyltransferase | | | | |

It is within the scope of the present invention that the genes presented in Table 1 are involved in phytoestrogen biosynthesis and elevating protein content pathways. Specifically, Glyma.01G239600, Glyma.01G228700, Glyma.10G010500.1, Glyma.11G010500, Glyma.13G173500, Glyma.14G005700, Glyma.17G064400, Glyma.16G149300, Glyma.11g011500, Glyma.02g005600, Glyma.17g030400 and Glyma19g030500 genes are involved in isoflavones and Coumestrol (CM) biosynthesis. Glyma.17G036400, Glyma.17G036300 and Glyma.19g135000 genes are involved in increasing protein content and accumulation in the Soybean plant and/or seed.

It is herein acknowledged that one of the potent phytoestrogens in Soybean is coumestrol (CM), which is a phytochemical in the class of coumestans, found mainly in Leguminosae (Fabaceae) plants. The CM biosynthesis pathway shares similarities with those of flavones and isoflavones.

The current invention is aimed at reducing or eliminating phytoestrogen level (such as isoflavones and Coumestrol) in the Soybean plant, using gene editing techniques (i.e. gRNA targeted CRISPR/Cas system).

According to further aspects of the present invention, Glyma.17G036400 (GmC/VIF2) and Glyma.17G036300 (GmCIF1) are involved in sugar metabolism (e.g. hexoses accumulation) and it is herein acknowledged that silencing their expression improves seed weight and protein content in Soybean. Without wishing to be bound by theory, it is acknowledged that the elevation of Cell wall invertase (CWI) by the silencing of its inhibitor isoforms (GmCIF1 and GmC/VIF2) may facilitate sucrose unloading and release more hexoses, which are required for starch and protein synthesis during seed maturation. In other words, suppression of extracellular invertase inhibitor gene expression improves seed weight and protein content in Soybean. With respect to Glyma.19g135000 gene, it encodes a fatty acid transporter and is involved in protein accumulation in seeds.

The present invention provides genomic sequence (as well as coding sequence and encoded polypeptide sequence) of the above genes (see Table 1) involved in phytoestrogen biosynthesis and protein accumulation in Soybean. The present invention further provides specific gRNA sequences for targeted in planta editing of each of these genes and thus manipulating their expression, resulting in the desirable traits or properties of reduced phytoestrogen and enhanced protein content in the Soybean plant and seed.

It is further within the scope that the present invention provides a method of identifying the specific gRNA sequences for each phytoestrogen synthesis gene and/or the genes allowing for higher protein content, paired with specific complementary PAMs. The present invention further discloses the characterization of a plurality of gRNAs directing the CRISPR/Cas system to cleave chromosomal strands coding for genes associated with phytoestrogen synthesis and/or higher protein content, including Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes (SEQ ID NO: 1, 220, 454, 674, 1014, 1305, 1491, 1798, 2090, 2200, 2296, 2630, 2810, 2973 and 3043, respectively).

Reference is now made to Table 1, presenting a list of Soybean genes involved in phytoestrogen synthesis and protein accumulation.

According to one embodiment, the present invention provides a method for eliminating or reducing phytoestrogen content in Soybean plants using gene editing.

According to a further embodiment, the present invention provides a method for increasing protein content in Soybean plants using gene editing, particularly via gRNA targeted endonuclease.

According to another embodiment, the present invention provides a method for eliminating or reducing phytoestrogen level and/or increasing protein content in Soybean plants, particularly via gRNA targeted endonuclease.

According some aspects, the method of the present invention comprising steps of;
a) selecting a gene involved in the phytoestrogen synthesis and/or in protein accumulation pathway (e.g. fatty acid transport and/or sugar metabolism) of the Soybean species;
b) synthesizing or designing a gRNA expression cassette corresponding to a targeted cleavage locus or region or domain within the selected gene (in the coding region or regulatory region) along the Soybean genome;
c) transforming said Soybean plant cells with CRISPR/Cas9 and the gRNA expression cassette;
d) culturing said transformed Soybean plant cells;
e) selecting said Soybean cells which express desired mutations in the editing target region, and
f) regenerating a plant from said transformed plant cell, plant cell nucleus, or plant tissue.

According to a further embodiment of the present invention, the gene involved in phytoestrogen synthesis of said Soybean species is selected from the group comprising Glyma.01G239600, Glyma.01G228700, Glyma.10G010500.1, Glyma.11G010500, Glyma.13G173500, Glyma.14G005700, Glyma.17G064400, Glyma.16G149300, Glyma.11g011500, Glyma.02g005600, Glyma.17g030400 and Glyma19g030500 (SEQ ID NO: 1, 220, 454, 674, 1014, 1305, 1491, 1798, 2630, 2810, 2973 and 3043, respectively).

According to a further embodiment of the present invention, the gene involved in protein content accumulation of said Soybean species is selected from the group comprising Glyma.17G036400, Glyma.17G036300 and Glyma.19g135000 (SEQ ID NO: 2090, 2200 and 2296, respectively).

According to a further embodiment of the present invention, the gRNAs and their corresponding PAMs are selected from a group comprising a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268 targeted to Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes, respectively.

It is further within the scope of the current invention that the target domain sequence is selected from the group comprising 1) a nucleic acid sequence encoding the polypeptide of any of the SEQ ID NO: 3, 222, 456, 676, 1016, 1307, 1493, 1800, 2092, 2202, 2298, 2632, 2812, 2975 and 3045, (2) a nucleic acid sequence comprising the sequence of any of SEQ ID NO: 1, 220, 454, 674, 1014, 1305, 1491, 1798, 2090, 2200, 2296, 2630, 2810, 2973 and 3043, and (3) a nucleic acid sequence having at least 80% sequence identity to at least 200 contiguous nucleotides of the nucleic acid sequence of SEQ ID NO: 1, 220, 454, 674, 1014, 1305, 1491, 1798, 2090, 2200, 2296, 2630, 2810, 2973 and 3043.

According to a further embodiment of the present invention, the transformation is carried out using Agrobacterium to deliver an expression cassette comprising a) a selection marker, b) a nucleotide sequence encoding one or more gRNA molecules comprising a DNA sequence which is complementary with a target domain sequence within a gene selected from Glyma.01G239600, Glyma.01G228700, Glyma.10G010500.1, Glyma.11G010500, Glyma.13 G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500, SEQ ID NO: 1, 220, 454, 674, 1014, 1305, 1491, 1798, 2090, 2200, 2296, 2630, 2810, 2973 and 3043, respectively.

According to a further embodiment of the present invention, the nucleotide sequence encoding a Cas molecule may be from, but not limited to Streptococcus pyogenes and/or Staphylococcus aureus (PAM sequences NGG and NNGRRT respectively). Other optional PAM within the scope of the present invention include, NNNNGATT (NmeCas9), NNAGAAW (StCas9), NAAAAC (TdCas9) and NNGRRT (SaCas9).

According to a further embodiment, the method of the present invention comprises administering a nucleic acid composition that comprises: a) a first nucleotide sequence encoding a gRNA molecule and b) a second nucleotide sequence encoding a Cas molecule.

According to a further embodiment of the present invention, the CRISPR/Cas system is delivered to the cell by a plant virus.

According to a further embodiment of the present invention, the Cas protein is selected from a group comprising, but not limited to, Cpf1, Cas9, Cas12, Cas13, Cas14, CasX or CasY.

It is further within the scope of the present invention to provide a method for increasing Soybean yield. The method comprising steps of: (a) introducing into a Soybean plant or a cell thereof (i) at least one RNA-guided endonuclease comprising at least one nuclear localization signal or nucleic acid encoding at least one RNA-guided endonuclease comprising at least one nuclear localization signal, (ii) at least one guide RNA or DNA encoding at least one guide RNA, and, optionally, (iii) at least one donor polynucleotide; and (b) culturing the Soybean plant or cell thereof such that each guide RNA directs an RNA-guided endonuclease to a targeted site in the chromosomal sequence where the RNA-guided endonuclease introduces a double-stranded break in the targeted site, and the double-stranded break is repaired by a DNA repair process such that the chromosomal sequence is modified, wherein the targeted site is located in at least one of Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes comprising a genomic sequence as set forth in SEQ ID NO: 1, 220, 454, 674, 1014, 1305, 1491, 1798, 2090, 2200, 2296, 2630, 2810, 2973 and 3043, respectively, or a functional variant thereof, and the chromosomal modification interrupts or interferes with transcription and/or translation of the at least one of the aforementioned genes.

According to a further embodiment of the present invention, the RNA-guided endonuclease is derived from a clustered regularly interspersed short palindromic repeats (CRISPR)/CRISPR-associated (Cas) system.

According to a further embodiment, the present invention discloses the gene editing of each of the following genes: Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 (SEQ ID NO: 1, 220, 454, 674, 1014, 1305, 1491, 1798, 2090, 2200, 2296, 2630, 2810, 2973 and 3043, respectively).

According to a further embodiment, the present invention provides a method that does not insert exogenous genetic material, but produces a non-naturally occurring Soybean plant or cell thereof.

According to a further embodiment, the present invention provides a method for manipulating Soybean protein, sugar and phytoestrogen synthesis genes by steps of:
(a) identifying at least one locus within a DNA sequence in a Soybean plant or a cell thereof for Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes (sequence comprising at least 80% sequence identity to SEQ ID NO: 1, 220, 454, 674, 1014, 1305, 1491, 1798, 2090, 2200, 2296, 2630, 2810, 2973 and 3043, respectively);
(b) identifying at least one custom endonuclease recognition sequence within the at least one locus of Glyma.01G239600, Glyma.01G228700, Glyma.10G010500.1, Glyma.11 G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes (sequence comprising at least 80% sequence identity to SEQ ID NO: 1, 220, 454, 674, 1014, 1305, 1491, 1798, 2090, 2200, 2296, 2630, 2810, 2973 and 3043, respectively); and
(e) identifying the Soybean plant, a cell thereof, or a progeny cell thereof as comprising a modification in a loci of at least one of Glyma.01G239600, Glyma.01G228700, Glyma.10G010500.1, Glyma.11 G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes (sequence comprising at least 80% sequence identity to SEQ ID NO: 1, 220, 454, 674, 1014, 1305, 1491, 1798, 2090, 2200, 2296, 2630, 2810, 2973 and 3043, respectively).

According to a further embodiment, the present invention provides a transgenic Soybean plant produced by the method as defined in any of the above.

It is further within the scope to provide a method for editing of genes involved in the protein, and/or phytoestrogen synthesis pathway, e.g. Glyma.01G239600, Glyma.01G228700, Glyma.10G010500.1, Glyma.11 G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes.

It is further within the scope of the present invention that the method as defined above comprises identifying and targeting the specific gRNA sequence for each individual gene defined in Table 1, constructing specific gRNAs for that region in each individual gene, thereby silencing (or alternatively enhancing) by use of gene editing technology the above-mentioned genes.

According to a further embodiment, the present invention provides a genome edited Soybean plant produced by the method as defined in any of the above, having elevated levels of protein as compared to control non edited Soybean plant.

According to a further embodiment, the present invention provides a genome edited Soybean plant produced by the method as defined in any of the above, having lower or decreased concentrations of phytoestrogen as compared to control non-edited Soybean plant.

As used herein the term “about” denotes ±25% of the defined amount or measure or value.

As used herein the term “similar” denotes a correspondence or resemblance range of about ±20%, particularly ±15%, more particularly about ±10% and even more particularly about ±5%.

As used herein the term “corresponding” generally means similar, analogous, like, alike, akin, parallel, identical, resembling or comparable. In further aspects it means having or participating in the same relationship (such as type or species, kind, degree, position, correspondence, or function). It further means related or accompanying. In some embodiments of the present invention it refers to plants of the same Soybean species or strain or variety or to sibling plant, or one or more individuals having one or both parents in common.

The term “corresponding” or “corresponding to” or “corresponding to nucleotide sequence” or “corresponding to position” as used herein, also refers in the context of the present invention to sequence homology or sequence identity. These terms relate to two or more nucleic acid or protein sequences, that are the same or have a specified percentage of amino acid residues or nucleotides that are the same, when compared and aligned for maximum correspondence, as measured using one of the available sequence comparison algorithms or by visual inspection. If two sequences, which are to be compared with each other, differ in length, sequence identity preferably relates to the percentage of the nucleotide residues of the shorter sequence, which are identical with the nucleotide residues of the longer sequence. As used herein, the percent of identity or homology between two sequences is a function of the number of identical positions shared by the sequences, taking into account the number of gaps, and the length of each gap, which needs to be introduced for optimal alignment of the two sequences. The comparison of sequences and determination of identity percent between two sequences can be accomplished using a mathematical algorithm as known in the relevant art. According to further aspects of the invention, the term “corresponding to the nucleotide sequence” or “corresponding to position”, refers to variants, homologues and fragments of the indicated nucleotide sequence, which possess or perform the same biological function or correlates with the same phenotypic characteristic of the indicated nucleotide sequence.

Another indication that two nucleic acid sequences are substantially identical or that a sequence is “corresponding to the nucleotide sequence” is that the two molecules hybridize to each other under stringent conditions. High stringency conditions, such as high hybridization temperature and low salt in hybridization buffers, permits only hybridization between nucleic acid sequences that are highly similar, whereas low stringency conditions, such as lower temperature and high salt, allows hybridization when the sequences are less similar.

In other embodiments of the invention, such substantially identical sequences refer to polynucleotide or amino acid sequences that share at least about 80% similarity or identity, preferably at least about 90% similarity or identity, alternatively, about 95%, 96%, 97%, 98% or 99% similarity or identity to the indicated polynucleotide or amino acid sequences.

According to other aspects of the invention, the term “corresponding” refers also to complementary sequences or base pairing such that when they are aligned antiparallel to each other, the nucleotide bases at each position in the sequences will be complementary. The degree of complementarity between two nucleic acid strands may vary.

A “plant” as used herein refers to any plant at any stage of development, particularly a seed plant. The term “plant” includes the whole plant or any parts or derivatives thereof.

The term “plant cell” used herein refers to a structural and physiological unit of a plant, comprising a protoplast and a cell wall. The plant cell may be in a form of an isolated single cell or a cultured cell.

The term “plant cell culture” as used herein means cultures of plant units such as, for example, protoplasts, regenerable cells, cell culture, cells, cells in plant tissues, pollen, pollen tubes, ovules, embryo sacs, zygotes and embryos at various stages of development, leaves, roots, root tips, anthers, meristematic cells, microspores, flowers, cotyledons, pistil, fruit, seeds, seed coat or any combination thereof.

The term “plant material” or “plant part” used herein refers to leaves, stems, roots, root tips, flowers or flower parts, fruits, pollen, egg cells, zygotes, seeds, seed coat, cuttings, cell or tissue cultures, or any other part or product of a plant or a combination thereof. It further refers to plant cells, seeds, plant protoplasts, plant cell tissue culture from which plants can be regenerated, plant callus or calli, meristematic cells, microspores, embryos, immature embryos, pollen, ovules, anthers, fruit, flowers, leaves, cotyledons, pistil, seeds, seed coat, roots, root tips and the like.

A “plant organ” as used herein means a distinct and visibly structured and differentiated part of a plant such as a root, stem, leaf, flower, flower bud, or embryo.

The term “Plant tissue” as used herein means a group of plant cells organized into a structural and functional unit. Any tissue of a plant in planta or in culture is included. This term includes, but is not limited to, whole plants, plant organs, plant seeds, tissue culture, protoplasts, meristematic cells, calli and any group of plant cells organized into structural and/or functional units. The use of this term in conjunction with, or in the absence of, any specific type of plant tissue as listed above or otherwise embraced by this definition is not intended to be exclusive of any other type of plant tissue.

As used herein, the term “progeny” or “progenies” refers in a non limiting manner to offspring or descendant plants. According to certain embodiments, the term “progeny” or “progenies” refers to plants developed or grown or produced from the disclosed or deposited seeds as detailed inter alia. The grown plants preferably have the desired traits of the disclosed or deposited seeds, i.e. mutation, preferable loss of function mutation, in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

As used herein the term “genetic modification” or “genome modification” refers hereinafter to genetic manipulation or modulation, which is the direct manipulation of an organism's genes using biotechnology. It also refers to a set of technologies used to change the genetic makeup of cells, including the transfer of genes within and across species, targeted mutagenesis and genome editing technologies to produce improved organisms. According to main embodiments of the present invention, modified Soybean plants free of or with reduced phytoestrogen content are generated using genome editing mechanism. This technique enables to achieve in planta modification of specific genes that relate to and/or control phytoestrogen biosynthesis and/or protein content in Soybean plant or seed.

The term “genome editing”, or “genome/genetic modification” or “genome engineering” generally refers hereinafter to a type of genetic engineering in which DNA is inserted, deleted, modified or replaced in the genome of a living organism. Unlike previous genetic engineering techniques that randomly insert genetic material into a host genome, genome editing targets the insertions to site specific locations. In the context of the present invention, the term also include base editing technique.

It is within the scope of the present invention that the common methods for such editing use engineered nucleases, or “molecular scissors”. These nucleases create site-specific double-strand breaks (DSBs) at desired locations in the genome. The induced double-strand breaks are repaired through nonhomologous end-joining (NHEJ) or homologous recombination (HR), resulting in targeted mutations (‘edits’). Families of engineered nucleases used by the current invention include, but are not limited to: meganucleases, zinc finger nucleases (ZFNs), transcription activator-like effector-based nucleases (TALEN), and the clustered regularly interspaced short palindromic repeats (CRISPR/Cas9) system.

The term “base editing” or “base-editing” in the context of the present invention refers to a genome editing approach that uses components from CRISPR systems together with other enzymes to directly introduce point mutations into cellular DNA or RNA without making double-stranded DNA breaks (DSBs). It is within the scope that DNA base editors comprise a catalytically disabled or inactivated nuclease, called herein nickase (nCas) fused to a nucleobase deaminase enzyme or a DNA glycosylase inhibitor. It is acknowledged that RNA base editors achieve analogous changes using components that target RNA. According to aspects of the present invention, base editors directly convert one base or base pair into another, enabling the efficient introduction of specific and precise point mutations in non-dividing cells without generating excess undesired editing byproducts such as indels, translocations, and rearrangements derived from DSBs created by nucleases such as Cas9 or any other Cas.

It is further within the scope of the current invention that DNA base editors (BEs) comprise fusions between a catalytically impaired Cas nuclease and a base-modification enzyme that operates on single-stranded DNA (ssDNA) but not double-stranded DNA (dsDNA). Without wishing to be bound by theory, it is noted that upon binding to its target locus in DNA, base pairing between the guide RNA and target DNA strand leads to displacement of a small segment of single-stranded DNA in an “R-loop”. DNA bases within this single-stranded DNA bubble are modified by the deaminase enzyme. To improve efficiency in eukaryotic cells, the catalytically disabled nuclease also generates a nick in the non-edited DNA strand, inducing cells to repair the non-edited strand using the edited strand as a template.

It is within the scope of the present invention that two classes of DNA base editor have been described: cytosine base editors (CBEs) which convert a CG base pair into a TA base pair, and adenine base editors (ABEs) which convert an AT base pair to a GC base pair. Together, CBEs and ABEs can mediate all four possible transition mutations (C to T, A to G, T to C, and G to A). In RNA, targeted adenosine conversion to inosine has been used in both antisense and Cas13-guided RNA-targeting methods.

Reference is now made to exemplary genome editing terms used by the current disclosure:

Genome Editing Glossary

| | |
|---|---|
| Cas = CRISPR-associated genes | Indel = insertion and/or deletion |
| Cas9, Csn1 = a CRISPR-associated protein | NHEJ = Non-Homologous End Joining |
| containing two nuclease domains, that is | PAM = Protospacer-Adjacent Motif |
| programmed by small RNAs to cleave DNA | RuvC = an endonuclease domain named for |
| crRNA = CRISPR RNA | an E. coli protein involved in DNA repair |
| dCAS9 = nuclease-deficient Cas9 | sgRNA = single guide RNA |
| DSB = Double-Stranded Break | tracrRNA, trRNA = trans-activating crRNA |
| gRNA = guide RNA | TALEN = Transcription-Activator Like |
| HDR = Homology-Directed Repair | Effector Nuclease |
| HNH = an endonuclease domain named | ZFN = Zinc-Finger Nuclease |
| for characteristic histidine and asparagine | |
| residues | |

According to specific aspects of the present invention, the CRISPR (Clustered Regularly Interspaced Short Palindromic Repeats) and CRISPR-associated (Cas) genes are used for the first time for generating genome modification in targeted genes in Soybean plant. It is herein acknowledged that the functions of CRISPR (Clustered Regularly Interspaced Short Palindromic Repeats) and CRISPR-associated (Cas) genes are essential in adaptive immunity in select bacteria and archaea, enabling the organisms to respond to and eliminate invading genetic material. These repeats were initially discovered in the 1980s in E. coli. Without wishing to be bound by theory, reference is now made to a type of CRISPR mechanism, in which invading DNA from viruses or plasmids is cut into small fragments and incorporated into a CRISPR locus comprising a series of short repeats (around 20 bps). The loci are transcribed, and transcripts are then processed to generate small RNAs (crRNA, namely CRISPR RNA), which are used to guide effector endonucleases that target invading DNA based on sequence complementarity.

According to further aspects of the invention, Cas protein, such as Cas9 (also known as Csn1) is required for gene silencing. Cas9 participates in the processing of crRNAs, and is responsible for the destruction of the target DNA. Cas9's function in both of these steps relies on the presence of two nuclease domains, a RuvC-like nuclease domain located at the amino terminus and a HNH-like nuclease domain that resides in the mid-region of the protein. To achieve site-specific DNA recognition and cleavage, Cas9 is complexed with both a crRNA and a separate trans-activating crRNA (tracrRNA or trRNA), that is partially complementary to the crRNA. The tracrRNA is required for crRNA maturation from a primary transcript encoding multiple pre-crRNAs. This occurs in the presence of RNase III and Cas9.

Without wishing to be bound by theory, it is herein acknowledged that during the destruction of target DNA, the HNH and RuvC-like nuclease domains cut both DNA strands, generating double-stranded breaks (DSBs) at sites defined by a 20-nucleotide target sequence within an associated crRNA transcript. The HNH domain cleaves the complementary strand, while the RuvC domain cleaves the noncomplementary strand.

It is further noted that the double-stranded endonuclease activity of Cas9 also requires that a short conserved sequence, (2-5 nts) known as protospacer-associated motif (PAM), follows immediately 3′- of the crRNA complementary sequence.

According to further aspects of the invention, a two-component system may be used by the current invention, combining trRNA and crRNA into a single synthetic single guide RNA (sgRNA) for guiding targeted gene alterations.

It is further within the scope that Cas9 nuclease variants include wild-type Cas9, Cas9D10A, or Cas having a mutation resulting in a nickase Cas (nCas).

Reference is now made to an example of CRISPR/Cas9 mechanism of action as depicted by Xie, Kabin, and Yinong Yang. “RNA-guided genome editing in plants using a CRISPR-Cas system.” Molecular plant 6.6 (2013): 1975-1983. As shown in this figure, the Cas9 endonuclease forms a complex with a chimeric RNA (called guide RNA or gRNA), replacing the crRNA-transcrRNA heteroduplex, and the gRNA could be programmed to target specific sites. The gRNA-Cas9 should comprise at least 15-base-pairing (gRNA seed region) without mismatch between the 5′-end of engineered gRNA and targeted genomic site, and an NGG motif (called protospacer-adjacent motif or PAM) that follows the base-pairing region in the complementary strand of the targeted DNA.

It is within the scope of the present invention that the Cas gene may be selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasY, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as Casa, (Cas-phi), Cast2f1, split Cas such as split Cas12a or split Cas9 or D10A or mutation resulting in a nickase Cas (nCas) and any combination thereof.

It is further within the scope of the present invention that the gRNA or sgRNA sequence comprises a 3′ Protospacer Adjacent Motif (PAM) selected from the group consisting of NGG (SpCas), NNNNGATT (NmeCas9), NNAGAAW (StCas9), NAAAAC (TdCas9), NNGRRT (SaCas9), TTTR PAM (Cas12f1) and TBN (Cas-phi).

A guide nucleic acid includes gRNA, gDNA, crRNA and crDNA. The term “meganucleases” as used herein refers hereinafter to endodeoxyribonucleases characterized by a large recognition site (double-stranded DNA sequences of 12 to 40 base pairs); as a result this site generally occurs only once in any given genome. Meganucleases are therefore considered to be the most specific naturally occurring restriction enzymes.

The term “protospacer adjacent motif” or “PAM” as used herein refers hereinafter to a 2-6 base pair DNA sequence immediately following the DNA sequence targeted by the Cas9 nuclease in the CRISPR bacterial adaptive immune system. PAM is a component of the invading virus or plasmid, but is not a component of the bacterial CRISPR locus. PAM is an essential targeting component which distinguishes bacterial self from non-self DNA, thereby preventing the CRISPR locus from being targeted and destroyed by nuclease.

The term “Next-generation sequencing” or “NGS” as used herein refers hereinafter to massively, parallel, high-throughput or deep sequencing technology platforms that perform sequencing of millions of small fragments of DNA in parallel. Bioinformatics analyses are used to piece together these fragments by mapping the individual reads to the reference genome.

The term “gene knockdown” as used herein refers hereinafter to an experimental technique by which the expression of one or more of an organism's genes is reduced. The reduction can occur through genetic modification, i.e. targeted genome editing or by treatment with a reagent such as a short DNA or RNA oligonucleotide that has a sequence complementary to either gene or an mRNA transcript. The reduced expression can be at the level of RNA or at the level of protein. It is within the scope of the present invention that the term gene knockdown also refers to a loss of function mutation and/or gene knockout mutation in which an organism's genes is made inoperative or nonfunctional.

The term “gene silencing” as used herein refers hereinafter to the regulation of gene expression in a cell to prevent the expression of a certain gene. Gene silencing can occur during either transcription or translation. In certain aspects of the invention, gene silencing is considered to have a similar meaning as gene knockdown. When genes are silenced, their expression is reduced. In contrast, when genes are knocked out, they are completely not expressed. Gene silencing may be considered a gene knockdown mechanism since the methods used to silence genes, such as RNAi, CRISPR, or siRNA, generally reduce the expression of a gene by at least 70% but do not completely eliminate it.

The term “loss of function mutation” as used herein refers to a type of mutation in which the altered gene product lacks the function of the wild-type gene. A synonyms of the term included within the scope of the present invention is null mutation.

The term “microRNAs” or “miRNAs” refers hereinafter to small non-coding RNAs that have been found in most of the eukaryotic organisms. They are involved in the regulation of gene expression at the post-transcriptional level in a sequence specific manner MiRNAs are produced from their precursors by Dicer-dependent small RNA biogenesis pathway. MiRNAs are candidates for studying gene function using different RNA-based gene silencing techniques. For example, artificial miRNAs (amiRNAs) targeting one or several genes of interest is a potential tool in functional genomics.

The term “in planta” means in the context of the present invention within the plant or plant cells. More specifically, it means introducing CRISPR/Cas complex into plant material comprising a tissue culture of several cells, a whole plant, or into a single plant cell, without introducing a foreign gene or a mutated gene. It also used to describe conditions present in a non-laboratory environment (e.g. in vivo).

The term “orthologue” as used herein refers hereinafter to one of two or more homologous gene sequences found in different species. In the context of the present invention it includes homologs, orthologs, paralogs and variants.

The term “functional variant” or “functional variant of a nucleic acid, polynucleotide, amino acid, polypeptide or protein sequence” as used herein, for example with reference to SEQ ID NOs: 1-3268, refers to a variant, homolog, orthologue or paralog of a sequence or part of a sequence which retains the biological function of the full non-variant allele and hence has the activity of the expressed gene or protein. A functional variant also comprises a variant of the gene of interest encoding a polypeptide which has sequence alterations that do not affect function of the resulting protein, for example, in non-conserved residues. Also encompassed is a variant that is substantially identical, i.e. has only some sequence variations, for example, in non-conserved residues, to the wild type nucleic acid or amino acid sequences of the alleles as shown herein, and is biologically active.

The term “variety” or “cultivar” used herein means a group of similar plants that by structural features and performance can be identified from other varieties within the same species.

The term “allele” used herein means any of one or more alternative or variant forms of a gene or a genetic unit at a particular locus, all of which alleles relate to one trait or characteristic at a specific locus. In a diploid cell of an organism, alleles of a given gene are located at a specific location, or locus (loci plural) on a chromosome. Alternative or variant forms of alleles may be the result of single nucleotide polymorphisms, insertions, inversions, translocations or deletions, or the consequence of gene regulation caused by, for example, by chemical or structural modification, transcription regulation or post-translational modification/regulation. An allele associated with a qualitative trait may comprise alternative or variant forms of various genetic units including those mat are identical or associated with a single gene or multiple genes or their products or even a gene disrupting or controlled by a genetic factor contributing to the phenotype represented by the locus. According to further embodiments, the term “allele” designates any of one or more alternative forms of a gene at a particular locus. Heterozygous alleles are two different alleles at the same locus. Homozygous alleles are two identical alleles at a particular locus. A wild type allele is a naturally occurring allele.

As used herein, the term “locus” (loci plural) means a specific place or places or region or a site on a chromosome where for example a gene or genetic marker element or factor is found. In specific embodiments, such a genetic element is contributing to a trait.

As used herein, the term “homozygous” refers to a genetic condition or configuration existing when two identical or like alleles reside at a specific locus, but are positioned individually on corresponding pairs of homologous chromosomes in the cell of a diploid organism.

Conversely, as used herein, the term “heterozygous” means a genetic condition or configuration existing when two different or unlike alleles reside at a specific locus, but are positioned individually on corresponding pairs of homologous chromosomes in the cell of a diploid organism.

As used herein, the phrase “genetic marker” or “molecular marker” or “biomarker” refers to a feature in an individual's genome e.g., a nucleotide or a polynucleotide sequence that is associated with one or more loci or trait of interest In some embodiments, a genetic marker is polymorphic in a population of interest, or the locus occupied by the polymorphism, depending on context. Genetic markers or molecular markers include, for example, single nucleotide polymorphisms (SNPs), indels (i.e. insertions deletions), simple sequence repeats (SSRs), restriction fragment length polymorphisms (RFLPs), random amplified polymorphic DNAs (RAFDs), cleaved amplified polymorphic sequence (CAPS) markers, Diversity Arrays Technology (DArT) markers, and amplified fragment length polymorphisms (AFLPs) or combinations thereof, among many other examples such as the DNA sequence per se. Genetic markers can, for example, be used to locate genetic loci containing alleles on a chromosome that contribute to variability of phenotypic traits. The phrase “genetic marker” or “molecular marker” or “biomarker” can also refer to a polynucleotide sequence complementary or corresponding to a genomic sequence, such as a sequence of a nucleic acid used as a probe or primer.

As used herein, the term “germplasm” refers to the totality of the genotypes of a population or other group of individuals (e.g., a species). The term “germplasm” can also refer to plant material; e.g., a group of plants that act as a repository for various alleles. Such germplasm genotypes or populations include plant materials of proven genetic superiority; e.g., for a given environment or geographical area, and plant materials of unknown or unproven genetic value; that are not part of an established breeding population and that do not have a known relationship to a member of the established breeding population.

The terms “hybrid”, “hybrid plant” and “hybrid progeny” used herein refers to an individual produced from genetically different parents (e.g., a genetically heterozygous or mostly heterozygous individual).

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. The term further refers hereinafter to the amount of characters which match exactly between two different sequences. Hereby, gaps are not counted and the measurement is relational to the shorter of the two sequences.

It is further within the scope that the terms “similarity” and “identity” additionally refer to local homology, identifying domains that are homologous or similar (in nucleotide and/or amino acid sequence). It is acknowledged that bioinformatics tools such as BLAST, SSEARCH, FASTA, and HMMER calculate local sequence alignments which identify the most similar region between two sequences. For domains that are found in different sequence contexts in different proteins, the alignment should be limited to the homologous domain, since the domain homology is providing the sequence similarity captured in the score. According to some aspects the term similarity or identity further includes a sequence motif, which is a nucleotide or amino-acid sequence pattern that is widespread and has, or is conjectured to have, a biological significance. Proteins may have a sequence motif and/or a structural motif, a motif formed by the three-dimensional arrangement of amino acids which may not be adjacent.

As used herein, the terms “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one embodiment, the nucleic acid is cDNA or coding DNA.

The terms “peptide”, “polypeptide” and “protein” are used interchangeably herein and refer to amino acids in a polymeric form of any length, linked together by peptide bonds.

According to other aspects of the invention, a “modified” or a “mutant” plant is a plant that has been altered compared to the naturally occurring wild type (WT) plant. Specifically, the endogenous nucleic acid sequences of each of the Soybean homologs (nucleic acid sequences comprising SEQ ID NO: 1, 2, 220, 221, 454, 455, 674, 675, 1014, 1015, 1305, 1306, 1491, 1492, 1798, 1799, 2090, 2091, 2200, 2201, 2296, 2297, 2630, 2631, 2810, 2811, 2973, 2974, 3043 and 3044) have been altered compared to wild type sequences using genome editing methods as described herein. This causes inactivation of the endogenous genes and thus disables their function (reduce or silence the expression or function of the endogenous genes). In alternative embodiments it may enhance the expression or function of the endogenous genes. Such plants have an altered phenotype and show decreased phytoestrogen content (decreased expression of genes involved in phytoestrogen biosynthesis) and/or increased protein content traits compared to wild type plants.

Therefore, the aforementioned traits are conferred by the presence of at least one mutated endogenous gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 in the Soybean plant genome which has been specifically targeted using genome editing technique (gRNA targeted endonuclease, such as the CRISPR/Cas system).

Main aspects of the invention involve targeted mutagenesis methods, specifically genome editing, and exclude embodiments that are solely based on generating plants by traditional breeding methods. In a further embodiment of the current invention, as explained herein, the trait of reduced phytoestrogen content and/or increased protein accumulation is not due to the presence of a transgene.

The loss of function mutation may be a deletion or insertion (“indels”) with reference the wild type Glyma.01G239600, Glyma.01G228700, Glyma.10G010500.1, Glyma.11 G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 allele sequence (SEQ ID NO: 1, 220, 454, 674, 1014, 1305, 1491, 1798, 2090, 2200, 2296, 2630, 2810, 2973 and 3043, respectively). The deletion may comprise 1-20 or more nucleotides, for example 1, 2, 3, 4, 5, 6, 7, 8, 9, 10, 1, 12, 13, 14, 15, 16, 17, 18 or 20 nucleotides or more in one or more strand. The insertion may comprise 1-20 or more nucleotides, for example 1, 2, 3, 4, 5, 6, 7, 8, 9, 10, 1, 12, 13, 14, 15, 16, 17, 18 or 20 or more nucleotides in one or more strand.

The plant of the invention includes plants wherein the plant is heterozygous for the each of the mutations. In a preferred embodiment however, the plant is homozygous for the mutations. Progeny that is also homozygous can be generated from these plants according to methods known in the art.

It is further within the scope that variants (homologs, paralogs or orthologues) of a particular nucleotide or amino acid sequence according to the various aspects of the invention will have at least about 50%-99%, for example at least 75%, for example at least 85%, 86%, 87%, 88%, 89%, 90%, 92%, 94%, 95%, 96%, 97%, 98% or 99% or more sequence identity to that particular non-variant nucleotide sequence of the allele as shown in SEQ ID NO: SEQ ID NO: 1, 220, 454, 674, 1014, 1305, 1491, 1798, 2090, 2200, 2296, 2630, 2810, 2973 and 3043, or amino acid sequence as shown in SEQ ID NO: 3, 222, 456, 676, 1016, 1307, 1493, 1800, 2092, 2202, 2298, 2632, 2812, 2975 and 3045. Sequence alignment programs to determine sequence identity are well known in the art.

Also, the various aspects of the invention encompass not only a nucleic acid or amino acid sequence encoding Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500, but also fragments thereof. By “fragment” is intended a portion of the nucleotide sequence or a portion of the amino acid sequence and hence of the protein encoded thereby. Fragments of a nucleotide sequence may encode protein fragments that retain the biological activity of the native protein, in this case improved domestication trait.

According to further embodiments of the present invention, DNA introduction into the plant cells can be done by Agrobacterium infiltration, virus based plasmids for delivery of the genome editing molecules and mechanical insertion of DNA (PEG mediated DNA transformation, biolistics, etc.).

In addition, it is within the scope of the present invention that the Cas9 protein is directly inserted together with a gRNA (ribonucleoprotein—RNP's) in order to bypass the need for in vivo transcription and translation of the Cas9+gRNA plasmid in planta to achieve gene editing.

It is also possible to create a genome edited plant and use it as a rootstock. Then, the Cas protein and gRNA can be transported via the vasculature system to the top of the plant and create the genome editing event in the scion.

It is within the scope of the present invention that the usage of CRISPR/Cas system for the generation of Soybean plants with reduced phytoestrogen content, allows the modification of predetermined specific DNA sequences without introducing foreign DNA into the genome by GMO techniques. According to one embodiment of the present invention, this is achieved by combining the Cas nuclease (e.g. Cas9, Cpf1 and the like) with a predefined guide RNA molecule (gRNA). The gRNA is complementary to a specific DNA sequence targeted for editing in the plant genome and which guides the Cas nuclease to a specific nucleotide sequence. The predefined gene specific gRNA's are cloned into the same plasmid as the Cas gene and this plasmid is inserted into plant cells. Insertion of the aforementioned plasmid DNA can be done, but not limited to, using different delivery systems, biological and/or mechanical, e.g. Agrobacterium infiltration, virus based plasmids for delivery of the genome editing molecules and mechanical insertion of DNA (PEG mediated DNA transformation, biolistics, etc.).

It is further within the scope of the present invention that upon reaching the specific predetermined DNA sequence, the Cas9 nuclease cleaves both DNA strands to create double stranded breaks leaving blunt ends. This cleavage site is then repaired by the cellular non homologous end joining DNA repair mechanism resulting in insertions or deletions which eventually create a mutation at the cleavage site. For example, it is acknowledged that a deletion form of the mutation consists of at least 1 base pair deletion. As a result of this base pair deletion the gene coding sequence is disrupted and the translation of the encoded protein is compromised either by a premature stop codon or disruption of a functional or structural property of the protein. Thus DNA is cut by the Cas9 protein and re-assembled by the cell's DNA repair mechanism.

It is further within the scope that improved health properties in Soybean plants (e.g. reduced phytoestrogen level and/or enhanced protein concentration or content) is herein produced by generating gRNA with homology to a specific site of predetermined genes in the Soybean genome i.e. Glyma.01 G239600, Glyma.01 G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes, sub cloning this gRNA into a plasmid containing the Cas9 gene, and insertion of the plasmid into the Soybean plant cells. In this way site specific mutations in the genes are generated thus effectively creating molecules with modulated expression, such as non-active molecules, resulting in reduction or elimination of phytoestrogens and increasing protein content of the genome edited Soybean plant.

According to one embodiment, the present invention provides a modified Soybean plant, plant part, or plant cell exhibiting reduced phytoestrogen content and/or increased protein content, wherein the modified Soybean plant, plant part, or plant cell comprises one or more gene editing derived mutations in at least one gene selected from Glyma.01G239600, Glyma.01G228700, Glyma.10G010500.1, Glyma.11 G010500, Glyma.13G173500, Glyma.14G005700, Glyma.17G064400, Glyma.16G149300, Glyma.17G036400, Glyma.17G036300, Glyma.19g135000, Glyma.11 g011500, Glyma.02g005600, Glyma.17g030400 and Glyma19g030500.

According to a further embodiment of the present invention, the modified Soybean plant, plant part, or plant cell as defined in any of the above is characterized by reduced phytoestrogen content and/or increased protein content, as compared to a Soybean plant absent of said one or more mutations.

According to a further embodiment of the present invention, the modified Soybean plant, plant part, or plant cell as defined in any of the above, wherein the plant, plant part, or plant cell is characterized by modulated or altered expression of said at least one gene as compared to a Soybean plant absent of said one or more mutations.

According to a further embodiment of the present invention, the modified Soybean plant, plant part, or plant cell as defined in any of the above, wherein the plant, plant part, or plant cell is characterized by reduced expression of said at least one gene as compared to a Soybean plant absent of said one or more mutations.

According to a further embodiment of the present invention, the modified Soybean plant, plant part, or plant cell as defined in any of the above, wherein the plant, plant part, or plant cell comprises one or more gene editing derived mutations in at least one gene selected from Glyma.01G239600, Glyma.01G228700, Glyma.10G010500.1, Glyma.11 G010500, Glyma.13G173500, Glyma.14G005700, Glyma.17G064400, Glyma.16G149300, Glyma.11g011500, Glyma.02g005600, Glyma.17g030400 and Glyma19g030500 conferring reduced phytoestrogen content to said plant, plant part, or plant cell.

According to a further embodiment of the present invention, the modified Soybean plant, plant part, or plant cell as defined in any of the above, wherein the plant, plant part, or plant cell comprises one or more gene editing derived mutations in at least one gene selected from Glyma.17G036400, Glyma.17G036300 and Glyma.19g135000 conferring increased protein content in the Soybean plant, plant part, or plant cell.

According to a further embodiment of the present invention, the modified Soybean plant, plant part, or plant cell as defined in any of the above, wherein the Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes comprise a genomic sequence as set forth in SEQ ID NO: 1, 220, 454, 674, 1014, 1305, 1491, 1798, 2090, 2200, 2296, 2630, 2810, 2973 and 3043, respectively, or a functional variant thereof.

According to a further embodiment of the present invention, the modified Soybean plant, plant part, or plant cell as defined in any of the above, wherein the functional variant has at least 80% sequence identity to said SEQ ID NO: 1, 220, 454, 674, 1014, 1305, 1491, 1798, 2090, 2200, 2296, 2630, 2810, 2973 and 3043.

According to a further embodiment of the present invention, the modified Soybean plant, plant part, or plant cell as defined in any of the above, wherein the sequence of a target editing region within the at least one gene is selected from the group comprising a) a nucleic acid sequence encoding the polypeptide comprising a sequence selected from the group comprising SEQ ID NO: 3, 222, 456, 676, 1016, 1307, 1493, 1800, 2092, 2202, 2298, 2632, 2812, 2975 and 3045, b) a nucleic acid sequence comprising a sequence selected from the group comprising SEQ ID NO: 1, 220, 454, 674, 1014, 1305, 1491, 1798, 2090, 2200, 2296, 2630, 2810, 2973 and 3043, and c) a nucleic acid sequence having at least 80% sequence identity to at least 200 contiguous nucleotides of a nucleic acid sequence selected from the group comprising SEQ ID NO: 1, 220, 454, 674, 1014, 1305, 1491, 1798, 2090, 2200, 2296, 2630, 2810, 2973 and 3043.

According to a further embodiment of the present invention, the modified Soybean plant, plant part, or plant cell as defined in any of the above, wherein the one or more mutations is introduced in planta using targeted genome modification.

According to a further embodiment of the present invention, the modified Soybean plant, plant part, or plant cell as defined in any of the above, wherein the one or more mutations is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

According to a further embodiment of the present invention, the modified Soybean plant, plant part, or plant cell as defined in any of the above, wherein said Cas gene is selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasY, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966 and any combination thereof.

According to a further embodiment of the present invention, the modified Soybean plant, plant part, or plant cell as defined in any of the above, wherein the at least one mutated gene is a CRISPR/Cas9-induced heritable mutated allele.

According to a further embodiment of the present invention, the modified Soybean plant, plant part, or plant cell as defined in any of the above, wherein the one or more mutations is selected from a silencing mutation, a knockdown mutation, a knockout mutation, a loss of function mutation, a downregulation mutation, an upregulation mutation or any combination thereof.

According to a further embodiment of the present invention, the modified Soybean plant, plant part, or plant cell as defined in any of the above, wherein the one or more mutations is in the coding region of said gene, a mutation in the regulatory region of said gene, or an epigenetic factor.

According to a further embodiment of the present invention, the modified Soybean plant, plant part, or plant cell as defined in any of the above, wherein said plant, plant part, or plant cell is homozygous for said at least one mutated gene.

According to a further embodiment of the present invention, the modified Soybean plant, plant part, or plant cell as defined in any of the above, wherein said plant, plant part, or plant cell is heterozygous for said at least one mutated gene.

According to a further embodiment of the present invention, the modified Soybean plant, plant part, or plant cell as defined in any of the above, wherein the one or more mutations is generated in planta via introduction of (a) Cas DNA and guide RNA (gRNA) sequence complementary to SEQ ID NO: 1, 220, 454, 674, 1014, 1305, 1491, 1798, 2090, 2200, 2296, 2630, 2810, 2973 and 3043, or (b) a ribonucleoprotein (RNP) complex comprising Cas protein and gRNA sequence complementary to SEQ ID NO: 1, 220, 454, 674, 1014, 1305, 1491, 1798, 2090, 2200, 2296, 2630, 2810, 2973 and 3043.

According to a further embodiment of the present invention, the modified Soybean plant, plant part, or plant cell as defined in any of the above, wherein said one or more mutations is generated in planta in said at least one Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes via introduction of (a) Cas DNA and guide RNA (gRNA) sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, respectively, and any combination thereof, or (b) a ribonucleoprotein (RNP) complex comprising Cas protein and gRNA sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, respectively, and any combination thereof.

According to a further embodiment of the present invention, the modified Soybean plant, plant part, or plant cell as defined in any of the above, wherein said gRNA sequence comprises a 3′ Protospacer Adjacent Motif (PAM) selected from NGG, NNGRRT, NNNNGATT, NNAGAAW and NAAAAC.

According to a further embodiment of the present invention, the modified Soybean plant, plant part, or plant cell as defined in any of the above, wherein the plant, plant part, or plant cell does not comprise a transgene.

According to a further embodiment of the present invention, the modified Soybean plant, plant part, or plant cell as defined in any of the above, wherein the plant part is a seed, tissue culture of regenerable cells, protoplasts, callus or pollen.

According to a further embodiment of the present invention, the modified Soybean plant, plant part, or plant cell as defined in any of the above, wherein the plant, plant part, or plant cell has a content of phytoestrogen that is reduced by about 10%, about 20%, about 30%, about 50%, about 60%, about 70%, about 80%, or about 90% compared to the content of phytoestrogen in a Soybean plant, plant part, or plant cell absent of said one or more mutations.

According to a further embodiment of the present invention, the modified Soybean plant, plant part, or plant cell as defined in any of the above, wherein the phytoestrogen is selected from isoflavone and/or Coumestrol.

According to a further embodiment of the present invention, the modified Soybean plant, plant part, or plant cell as defined in any of the above, wherein the plant, plant part, or plant cell has a protein content that is elevated by about 10%, about 20%, about 30%, about 50%, about 60%, about 70%, about 80%, or about 90% compared to the protein content in a Soybean plant, plant part, or plant cell absent of said one or more mutations.

It is further within the scope of the present invention to provide Soybean seed, pollen, plant parts or progeny of the modified Soybean plant as defined in any of the above, wherein said seed, pollen, plant parts or progeny comprises said one or more gene editing derived mutations in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

It is further within the scope of the present invention to provide food and non-food products incorporating the modified Soybean plant, plant part or plant cell as defined in any of the above.

It is further within the scope of the present invention to provide food and non-food products incorporating a seed of a Soybean plant as defined in any of the above.

It is further within the scope of the present invention to disclose a method for producing a modified Soybean plant, plant part, or plant cell exhibiting reduced phytoestrogen content and/or increased protein content using targeted genome modification, comprising steps of: (a) identifying a gene involved in phytoestrogen synthesis and/or increased protein content of a Soybean plant species; (b) synthesizing or designing a gRNA expression cassette corresponding to a target editing region along the Soybean genome, within the identified gene locus; (c) transforming said Soybean plant, plant part, or plant cell with CRISPR/Cas9 system; (d) screening the genome of said transformed plant, plant part, or plant cell for induced targeted mutation, e.g. loss of function mutation in the identified gene involved in phytoestrogen synthesis and/or increased protein content; (e) selecting said Soybean plant, plant part, or plant cell expressing desired one or more mutations in the editing target region, said mutations confer reduced phytoestrogen content and/or increased protein content; and (f) optionally, regenerating a plant from said transformed plant, plant part, or plant cell plant cell nucleus, or plant tissue and optionally screening said regenerated plants for a Soybean plant, with decreased phytoestrogen content and/or increased protein content.

It is further within the scope of the present invention to disclose the method as defined in any of the above, wherein the gRNA and its corresponding PAM is complementary to a sequence of a gene selected from a group comprising at least one of Glyma.01G239600, Glyma.01G228700, Glyma.10G010500.1, Glyma.11 G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

It is further within the scope of the present invention to disclose a method for producing a modified Soybean plant, plant part, or plant cell exhibiting reduced phytoestrogen content and/or increased protein content using targeted genome modification comprising introducing into the genome of the plant, plant part, or plant cell one or more mutations in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

It is further within the scope of the present invention to disclose the method as defined in any of the above, wherein the plant, plant part, or plant cell is characterized by reduced phytoestrogen content and/or increased protein content, as compared to a Soybean plant absent of said one or more mutations.

It is further within the scope of the present invention to disclose the method as defined in any of the above, wherein the plant, plant part, or plant cell is characterized by modulated or altered expression of said at least one gene as compared to a Soybean plant absent of said one or more mutations.

It is further within the scope of the present invention to disclose the method as defined in any of the above, wherein the plant, plant part, or plant cell is characterized by reduced expression of said at least one gene as compared to a Soybean plant absent of said one or more mutations.

It is further within the scope of the present invention to disclose the method as defined in any of the above, wherein the plant, plant part, or plant cell comprises one or more gene editing derived mutations in at least one gene selected from Glyma.01G239600, Glyma.01G228700, Glyma.10G010500.1, Glyma.11 G010500, Glyma.13G173500, Glyma.14G005700, Glyma.17G064400, Glyma.16G149300, Glyma.11g011500, Glyma.02g005600, Glyma.17g030400 and Glyma19g030500 conferring reduced phytoestrogen content to said plant, plant part, or plant cell.

It is further within the scope of the present invention to disclose the method as defined in any of the above, wherein the plant, plant part, or plant cell comprises one or more gene editing derived mutations in at least one gene selected from Glyma.17G036400, Glyma.17G036300 and Glyma.19g135000 conferring increased protein content in the Soybean plant, plant part, or plant cell.

It is further within the scope of the present invention to disclose the method as defined in any of the above, wherein the Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes comprise a genomic sequence as set forth in SEQ ID NO: 1, 220, 454, 674, 1014, 1305, 1491, 1798, 2090, 2200, 2296, 2630, 2810, 2973 and 3043, respectively, or a functional variant thereof.

It is further within the scope of the present invention to disclose the method as defined in any of the above, wherein the functional variant has at least 80% sequence identity to said SEQ ID NO: 1, 220, 454, 674, 1014, 1305, 1491, 1798, 2090, 2200, 2296, 2630, 2810, 2973 and 3043.

It is further within the scope of the present invention to disclose the method as defined in any of the above, wherein the sequence of a target editing region within the at least one gene is selected from the group comprising a) a nucleic acid sequence encoding the polypeptide comprising a sequence selected from the group comprising SEQ ID NO: 3, 222, 456, 676, 1016, 1307, 1493, 1800, 2092, 2202, 2298, 2632, 2812, 2975 and 3045, b) a nucleic acid sequence comprising a sequence selected from the group comprising SEQ ID NO: 1, 220, 454, 674, 1014, 1305, 1491, 1798, 2090, 2200, 2296, 2630, 2810, 2973 and 3043, and c) a nucleic acid sequence having at least 80% sequence identity to at least 200 contiguous nucleotides of a nucleic acid sequence selected from the group comprising SEQ ID NO: 1, 220, 454, 674, 1014, 1305, 1491, 1798, 2090, 2200, 2296, 2630, 2810, 2973 and 3043.

It is further within the scope of the present invention to disclose the method as defined in any of the above, wherein the one or more mutations is introduced in planta using targeted genome modification.

It is further within the scope of the present invention to disclose the method as defined in any of the above, wherein the one or more mutations is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

It is further within the scope of the present invention to disclose the method as defined in any of the above, wherein said Cas gene is selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasY, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966 and any combination thereof.

It is further within the scope of the present invention to disclose the method as defined in any of the above, wherein the at least one mutated gene is a CRISPR/Cas9-induced heritable mutated allele.

It is further within the scope of the present invention to disclose the method as defined in any of the above, wherein the one or more mutations is selected from a silencing mutation, a knockdown mutation, a knockout mutation, a loss of function mutation, a downregulation mutation, an upregulation mutation or any combination thereof.

It is further within the scope of the present invention to disclose the method as defined in any of the above, wherein the one or more mutations is in the coding region of said gene, a mutation in the regulatory region of said gene, or an epigenetic factor.

It is further within the scope of the present invention to disclose the method as defined in any of the above, wherein said plant, plant part, or plant cell is homozygous for said at least one mutated gene.

It is further within the scope of the present invention to disclose the method as defined in any of the above, wherein said plant, plant part, or plant cell is heterozygous for said at least one mutated gene.

It is further within the scope of the present invention to disclose the method as defined in any of the above, wherein the one or more mutations is generated in planta via introduction of (a) Cas DNA and guide RNA (gRNA) sequence complementary to SEQ ID NO: 1, 220, 454, 674, 1014, 1305, 1491, 1798, 2090, 2200, 2296, 2630, 2810, 2973 and 3043, or (b) a ribonucleoprotein (RNP) complex comprising Cas protein and gRNA sequence complementary to SEQ ID NO: 1, 220, 454, 674, 1014, 1305, 1491, 1798, 2090, 2200, 2296, 2630, 2810, 2973 and 3043.

It is further within the scope of the present invention to disclose the method as defined in any of the above, wherein said one or more mutations is generated in planta in said at least one Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes via introduction of (a) Cas DNA and guide RNA (gRNA) sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, respectively, or (b) a ribonucleoprotein (RNP) complex comprising Cas protein and gRNA sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, respectively.

It is further within the scope of the present invention to disclose the method as defined in any of the above, wherein said gRNA sequence comprises a 3′ Protospacer Adjacent Motif (PAM) selected from NGG, NNGRRT, NNNNGATT, NNAGAAW and NAAAAC.

It is further within the scope of the present invention to disclose the method as defined in any of the above, wherein the transformation is carried out to deliver an expression cassette comprising a) a selection marker, b) a nucleotide sequence encoding one or more gRNA molecules comprising a sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, and c) a nucleotide sequence encoding a Cas molecule.

It is further within the scope of the present invention to disclose the method as defined in any of the above, wherein the method comprises administering to the Soybean plant, plant part, or plant cell a nucleic acid composition comprising: a) a first nucleotide sequence encoding a gRNA molecule, and b) a second nucleotide sequence encoding the Cas molecule.

It is further within the scope of the present invention to disclose the method as defined in any of the above, wherein the CRISPR/Cas system is delivered to the cell by a plant virus.

It is further within the scope of the present invention to disclose the method as defined in any of the above, wherein the method comprises steps of (a) introducing into a Soybean plant or plant part or cell thereof (i) at least one RNA-guided endonuclease comprising at least one nuclear localization signal or nucleic acid encoding at least one RNA-guided endonuclease comprising at least one nuclear localization signal, (ii) at least one guide RNA (gRNA) or DNA encoding at least one gRNA, and, optionally, (iii) at least one donor polynucleotide; and (b) culturing the Soybean plant or plant part or cell thereof such that each gRNA directs an RNA-guided endonuclease to a target site in the chromosomal sequence where the RNA-guided endonuclease introduces a double-stranded break in the target site, and the double-stranded break is repaired by a DNA repair process such that the chromosomal sequence is modified, wherein the target site is located in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 and the chromosomal modification interrupts or interferes with transcription and/or translation of the at least one gene.

It is further within the scope of the present invention to disclose the method as defined in any of the above, wherein the RNA-guided endonuclease is derived from a clustered regularly interspersed short palindromic repeats (CRISPR)/CRISPR-associated (Cas) system.

It is further within the scope of the present invention to disclose the method as defined in any of the above, wherein introduction of a genome modification does not insert exogenous genetic material, and produces a non-naturally occurring or modified Soybean plant, plant part or cell thereof.

It is further within the scope of the present invention to disclose the method as defined in any of the above, further comprises steps of (a) identifying at least one locus within a DNA sequence of a Soybean plant or a cell thereof selected from Glyma.01G239600, Glyma.01G228700, Glyma.10G010500.1, Glyma.11 G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes; (b) identifying at least one custom endonuclease recognition sequence within the at least one locus of the gene; and (c) identifying the Soybean plant, a cell thereof, or a progeny cell thereof as comprising a modification in the loci of the at least one gene, said modification confers modulated or altered expression, e.g. silencing, of at least one gene selected from Glyma.01G239600, Glyma.01G228700, Glyma.10G010500.1, Glyma.11 G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

It is further within the scope of the present invention to disclose the method as defined in any of the above, wherein the plant, plant part, or plant cell does not comprise a transgene.

It is further within the scope of the present invention to disclose the method as defined in any of the above, wherein the plant part is a seed, tissue culture of regenerable cells, protoplasts, callus or pollen.

It is further within the scope of the present invention to disclose the method as defined in any of the above, wherein the plant, plant part, or plant cell has a content of phytoestrogen that is reduced by about 10%, about 20%, about 30%, about 50%, about 60%, about 70%, about 80%, or about 90% compared to the content of phytoestrogen in a Soybean plant, plant part, or plant cell absent of said one or more mutations.

It is further within the scope of the present invention to disclose the method as defined in any of the above, wherein the phytoestrogen is selected from isoflavone and/or Coumestrol.

It is further within the scope of the present invention to disclose the method as defined in any of the above, wherein the plant, plant part, or plant cell has a protein content that is elevated by about 10%, about 20%, about 30%, about 50%, about 60%, about 70%, about 80%, or about 90% compared to the protein content in a Soybean plant, plant part, or plant cell absent of said one or more mutations.

It is further within the scope of the present invention to disclose the method as defined in any of the above, wherein the method further comprises steps of analyzing the Soybean plants or progeny thereof, plant part, or plant cell by a) isolating genomic DNA from the mutated plant material or its progeny; and b) amplifying segments of a gene involved in phytoestrogen synthesis and/or a gene associated with protein accumulation such as fatty acid transport or sugar metabolism, in the isolated genomic DNA, using primers specific to at least one of Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes or to the DNA sequences adjacent to the at least one gene.

According to a further embodiment, the present invention provides a modified Soybean seed, pollen, plant parts or progeny with reduced phytoestrogen content and/or increased protein content, produced by the method as defined in any of the above, wherein said seed, pollen, plant parts or progeny comprises said one or more gene editing derived mutations in at least one gene selected from Glyma.01G239600, Glyma.01 G228700, Glyma.10G010500.1, Glyma.11 G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

It is further within the scope of the present invention to disclose a method for reducing phytoestrogen content and/or increasing protein content in a Soybean plant, plant part or plant cell, using targeted genome modification, comprising introducing into the genome of the plant, plant part, or plant cell one or more mutations in at least one gene selected from Glyma.01G239600, Glyma.01G228700, Glyma.10G010500.1, Glyma.11 G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

It is also within the scope of the present invention to disclose a method for increasing Soybean yield using targeted genome modification, comprising introducing into the genome of the Soybean plant, plant part, or plant cell one or more mutations in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

It is further within the scope of the present invention to disclose the method as defined in any of the above, wherein the plant part is selected from the group consisting of seeds, pollen, plant cells, or plant tissue.

It is further within the scope of the present invention to disclose the method as defined in any of the above, wherein the plant, plant part, or plant cell comprises one or more gene editing derived mutations in at least one gene selected from Glyma.01G239600, Glyma.01G228700, Glyma.10G010500.1, Glyma.11 G010500, Glyma.13G173500, Glyma.14G005700, Glyma.17G064400, Glyma.16G149300, Glyma.11g011500, Glyma.02g005600, Glyma.17g030400 and Glyma19g030500 conferring reduced phytoestrogen content to said plant, plant part, or plant cell.

It is further within the scope of the present invention to disclose the method as defined in any of the above, wherein the plant, plant part, or plant cell comprises one or more gene editing derived mutations in at least one gene selected from Glyma.17G036400, Glyma.17G036300 and Glyma.19g135000 conferring increased protein content in the Soybean plant, plant part, or plant cell.

It is further within the scope of the present invention to disclose the method as defined in any of the above, wherein the Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes comprise a genomic sequence as set forth in SEQ ID NO: 1, 220, 454, 674, 1014, 1305, 1491, 1798, 2090, 2200, 2296, 2630, 2810, 2973 and 3043, respectively, or a functional variant thereof.

It is further within the scope of the present invention to disclose the method as defined in any of the above, wherein the functional variant has at least 80% sequence identity to said SEQ ID NO: 1, 220, 454, 674, 1014, 1305, 1491, 1798, 2090, 2200, 2296, 2630, 2810, 2973 and 3043.

It is further within the scope of the present invention to disclose the method as defined in any of the above, wherein said one or more mutations is generated in planta in said at least one Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes via introduction of (a) Cas DNA and guide RNA (gRNA) sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, respectively, or (b) a ribonucleoprotein (RNP) complex comprising Cas protein and gRNA sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, respectively.

According to another embodiment, the present invention provides a construct comprising or encoding (a) a nucleotide sequence encoding a Cas molecule and one or more gRNA molecules comprising a sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042, SEQ ID NO: 3046-3268, and any combination thereof, or (b) a ribonucleoprotein (RNP) complex comprising Cas protein and one or more gRNA molecules comprising a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, and any combination thereof.

It is also within the scope of the present invention to disclose a method of modifying the phytoestrogen content and/or protein content of a Soybean plant, plant part, or plant cell, comprising introducing into a Soybean plant, plant part, or plant cell the construct as defined above.

It is also within the scope of the present invention to disclose an isolated polynucleotide sequence comprising at least 80% sequence identity to a polynucleotide sequence selected from the group consisting of SEQ ID NO: 1, 2, 220, 221, 454, 455, 674, 675, 1014, 1015, 1305, 1306, 1491, 1492, 1798, 1799, 2090, 2091, 2200, 2201, 2296, 2297, 2630, 2631, 2810, 2811, 2973, 2974, 3043 and 3044.

It is also within the scope of the present invention to disclose an isolated polypeptide sequence comprising at least 80% sequence identity to SEQ ID NO: 3, 222, 456, 676, 1016, 1307, 1493, 1800, 2092, 2202, 2298, 2632, 2812, 2975 and 3045.

According to a further embodiment, the present invention discloses a use of a nucleic acid sequence comprising at least 80% sequence identity to a sequence as set forth in at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2633-2809, SEQ ID NO: 2813-2972, SEQ ID NO: 2976-3042 or SEQ ID NO: 3046-3268 and any combination thereof for targeted gene editing of at least one Soybean gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.11g011500, Glyma.02g005600, Glyma.17g030400 and Glyma19g030500, respectively, said gene editing confers reduced phytoestrogen content in a Soybean plant, plant part or plant cell.

According to a further embodiment, the present invention discloses a use of a nucleic acid sequence comprising at least 80% sequence identity to a sequence as set forth in at least one of SEQ ID NO: 2093-2199, SEQ ID NO: 2203-2295, SEQ ID NO: 2299-2629 and any combination thereof, for targeted gene editing of at least one Soybean gene selected from Glyma.17G036400, Glyma.17G036300, Glyma.19g135000, respectively, said gene editing confers increased protein content in a Soybean plant, plant part or plant cell.

1. A modified Soybean plant, plant part, or plant cell, wherein the modified Soybean plant, plant part, or plant cell comprises one or more gene editing derived mutations in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.11g011500, Glyma.02g005600, Glyma.17g030400 and Glyma19g030500, conferring reduced phytoestrogen content to said plant, plant part, or plant cell, and/or one or more gene editing derived mutations in at least one gene selected from Glyma.17G036400, Glyma.17G036300 and Glyma.19g135000, conferring increased protein content to the Soybean plant, plant part, or plant cell.

2. The modified Soybean plant, plant part, or plant cell according to claim 1, wherein the plant, plant part, or plant cell is characterized by at least one of:
a. reduced phytoestrogen content and/or increased protein content, as compared to a Soybean plant absent of said one or more mutations;
b. modulated or altered expression of said at least one gene as compared to a Soybean plant absent of said one or more mutations; and
c. reduced expression of said at least one gene as compared to a Soybean plant absent of said one or more mutations.

3-6. (canceled)

7. The modified Soybean plant, plant part, or plant cell according to claim 1, wherein the Glyma.01G239600, Glyma.01G228700, Glyma.10G010500.1, Glyma.11G010500, Glyma.13 G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes comprise a genomic sequence having at least 80% sequence identity to SEQ ID NO: 1, 220, 454, 674, 1014, 1305, 1491, 1798, 2090, 2200, 2296, 2630, 2810, 2973 and 3043, respectively.

8. (canceled)

9. The modified Soybean plant, plant part, or plant cell according to claim 1, wherein the sequence of a target editing region within the at least one gene is selected from the group comprising a) a nucleic acid sequence encoding the polypeptide comprising a sequence selected from the group comprising SEQ ID NO: 3, 222, 456, 676, 1016, 1307, 1493, 1800, 2092, 2202, 2298, 2632, 2812, 2975 and 3045, b) a nucleic acid sequence comprising a sequence selected from the group comprising SEQ ID NO: 1, 220, 454, 674, 1014, 1305, 1491, 1798, 2090, 2200, 2296, 2630, 2810, 2973 and 3043, and c) a nucleic acid sequence having at least 80% sequence identity to at least 200 contiguous nucleotides of a nucleic acid sequence selected from the group comprising SEQ ID NO: 1, 220, 454, 674, 1014, 1305, 1491, 1798, 2090, 2200, 2296, 2630, 2810, 2973 and 3043.

10-18. (canceled)

19. The modified Soybean plant, plant part, or plant cell according to claim 1, wherein said one or more mutations is generated in planta in said at least one Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes via introduction of (a) Cas DNA and guide RNA (gRNA) sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, respectively, and any combination thereof, or (b) a ribonucleoprotein (RNP) complex comprising Cas protein and gRNA sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, respectively, and any combination thereof.

20-22. (canceled)

23. The modified Soybean plant, plant part, or plant cell according to claim 1, wherein (a) the plant, plant part, or plant cell comprising the one or more gene editing derived mutations in the at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.11g011500, Glyma.02g005600, Glyma.17g030400 and Glyma19g030500, has a content of phytoestrogen that is reduced by about 10%, about 20%, about 30%, about 50%, about 60%, about 70%, about 80%, or about 90% compared to the content of phytoestrogen in a Soybean plant, plant part, or plant cell absent of said one or more mutations, and/or (b) the plant, plant part, or plant cell comprising the one or more gene editing derived mutations in the at least one gene selected from Glyma.17G036400, Glyma.19g135000 and Glyma.17G036300, has a protein content that is elevated by about 10%, about 20%, about 30%, about 50%, about 60%, about 70%, about 80%, or about 90% compared to the protein content in a Soybean plant, plant part, or plant cell absent of said one or more mutations.

24. The modified Soybean plant, plant part, or plant cell according to claim 23, wherein the phytoestrogen is selected from isoflavone and/or Coumestrol.

25. (canceled)

26. Soybean seed, tissue culture of regenerable cells, protoplasts, callus, pollen, plant parts or progeny of the modified Soybean plant of claim 1.

27. Food and non-food products incorporating the modified Soybean plant, plant part or plant cell or a seed of a Soybean plant of claim 1.

28. (canceled)

29. A method for producing a modified Soybean plant, plant part, or plant cell exhibiting reduced phytoestrogen content and/or increased protein content using targeted genome modification comprising steps of:
a. identifying a gene involved in phytoestrogen synthesis or increased protein content of a Soybean plant species;
b. synthesizing or designing a gRNA expression cassette corresponding to a target editing region along the Soybean genome, within the identified gene locus;
c. transforming said Soybean plant, plant part, or plant cell with CRISPR/Cas9 system;
d. screening the genome of said transformed plant, plant part, or plant cell for induced targeted mutation, e.g. loss of function mutation in the identified gene involved in phytoestrogen synthesis or increased protein content;
e. selecting said Soybean plant, plant part, or plant cell expressing desired one or more mutations in the editing target region, said mutations confer reduced phytoestrogen content or increased protein content; and
f. optionally, regenerating a plant from said transformed plant, plant part, or plant cell plant cell nucleus, or plant tissue and optionally screening said regenerated plants for a Soybean plant, with decreased phytoestrogen content or increased protein content.

30. The method according to claim 29, wherein the gRNA and its corresponding PAM is complementary to a sequence of a gene involved in phytoestrogen synthesis selected from a group comprising at least one of Glyma.01G239600, Glyma.01G228700, Glyma.10G010500.1, Glyma.11G010500, Glyma.13G173500, Glyma.14G005700, Glyma.17G064400, Glyma.16G149300, Glyma.11g011500, Glyma.02g005600, Glyma.17g030400 and Glyma19g030500 or to a sequence of a gene involved in increased protein content selected from a group comprising at least one of Glyma.17G036400, Glyma.19g135000 and Glyma.17G036300.

31. (canceled)

32. The method according to according to claim 29, wherein the plant, plant part, or plant cell is characterized by at least one of:
a. reduced phytoestrogen content and/or increased protein content, as compared to a Soybean plant absent of said one or more mutations;
b. modulated or altered expression of said at least one gene as compared to a Soybean plant absent of said one or more mutations; and
c. reduced expression of said at least one gene as compared to a Soybean plant absent of said one or more mutations.

33-36. (canceled)

37. The method according to claim 29, wherein the Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes comprise a genomic sequence having at least 80% sequence identity to SEQ ID NO: 1, 220, 454, 674, 1014, 1305, 1491, 1798, 2090, 2200, 2296, 2630, 2810, 2973 and 3043, respectively.

38-48. (canceled)

49. The method according to claim 29, wherein said one or more mutations is generated in planta in said at least one Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500 genes via introduction of (a) Cas DNA and guide RNA (gRNA) sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, respectively, or (b) a ribonucleoprotein (RNP) complex comprising Cas protein and gRNA sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, respectively.

50-56. (canceled)

57. The method according to claim 29, further comprises steps of
(a) identifying at least one locus within a DNA sequence of a Soybean plant or a cell thereof selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.11g011500, Glyma.02g005600, Glyma.17g030400 and Glyma19g030500 genes, and/or within a DNA sequence of a Soybean plant or a cell thereof selected from Glyma.17G036400, Glyma.19g135000 and Glyma.17G036300;
(b) identifying at least one custom endonuclease recognition sequence within the at least one locus of the gene; and
(c) identifying the Soybean plant, a cell thereof, or a progeny cell thereof as comprising a modification in the loci of the at least one gene, said modification confers modulated or altered expression, e.g. silencing, of at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.11g011500, Glyma.02g005600, Glyma.17g030400 and Glyma19g030500 and/or at least one gene selected from Glyma.17G036400, Glyma.19g135000 and Glyma.17G036300.

58-59. (canceled)

60. The method according to claim 29, wherein (a) the plant, plant part, or plant cell comprising one or more mutations introduced by targeted genome modification in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.11g011500, Glyma.02g005600, Glyma.17g030400 and Glyma19g030500, has a content of phytoestrogen that is reduced by about 10%, about 20%, about 30%, about 50%, about 60%, about 70%, about 80%, or about 90% compared to the content of phytoestrogen in a Soybean plant, plant part, or plant cell absent of said one or more mutations, and/or wherein the plant, plant part, or plant cell comprising one or more mutations introduced by targeted genome modification in at least one gene selected from Glyma.17G036400, Glyma.19g135000 and Glyma.17G036300, has a protein content that is elevated by about 10%, about 20%, about 30%, about 50%, about 60%, about 70%, about 80%, or about 90% compared to the protein content in a Soybean plant, plant part, or plant cell absent of said one or more mutations.

61-63. (canceled)

64. A modified Soybean seed, pollen, plant parts or progeny with reduced phytoestrogen content and/or increased protein content, produced by the method according to claim 29, wherein said seed, pollen, plant parts or progeny with reduced phytoestrogen content comprises said one or more gene editing derived mutations in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.11g011500, Glyma.02g005600, Glyma.17g030400 and Glyma19g030500, and said seed, pollen, plant parts or progeny with increased protein content comprises said one or more gene editing derived mutations in at least one gene selected from Glyma.17G036400, Glyma.19g135000 and Glyma.17G036300.

65. The method according to claim 29, wherein said method is useful for (a) reducing phytoestrogen content and/or increasing protein content in a Soybean plant, plant part or plant cell, using targeted genome modification, comprising introducing into the genome of the plant, plant part, or plant cell one or more mutations in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.11g011500, Glyma.02g005600, Glyma.17g030400 and Glyma19g030500, conferring reduced phytoestrogen content, and/or introducing into the genome of the plant, plant part, or plant cell one or more mutations in at least one gene selected from Glyma.17G036400, Glyma.19g135000 and Glyma.17G036300, conferring increased protein content; and/or (b) increasing Soybean yield using targeted genome modification, comprising introducing into the genome of the Soybean plant, plant part, or plant cell one or more mutations in at least one gene selected from Glyma.01G239600, Glyma.01G228700, Glyma.10G010500.1, Glyma.11G010500, Glyma.13G173500, Glyma.14G005700, Glyma.17G064400, Glyma.16G149300, Glyma.17G036400, Glyma.17G036300, Glyma.19g135000, Glyma.11g011500, Glyma.02g005600, Glyma.17g030400 and Glyma19g030500.

66-72. (canceled)

73. A construct comprising or encoding (a) a nucleotide sequence encoding a Cas molecule and one or more gRNA molecules comprising a sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042, SEQ ID NO: 3046-3268, and any combination thereof, or (b) a ribonucleoprotein (RNP) complex comprising Cas protein and one or more gRNA molecules comprising a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2093-2199, SEQ ID NO: 2203-2295, SEQ ID NO: 2299-2629, SEQ ID NO: 2633-2809, SEQ ID NO: 2813-2972, SEQ ID NO: 2976-3042 or SEQ ID NO: 3046-3268, and any combination thereof.

74. A method of modifying the phytoestrogen content or protein content of a Soybean plant, plant part, or plant cell, comprising introducing into a Soybean plant, plant part, or plant cell the construct of claim 73 comprising or encoding (a) (i) a nucleotide sequence encoding a Cas molecule and one or more gRNA molecules comprising a sequence selected from the group consisting of a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2633-2809, SEQ ID NO: 2813-2972, SEQ ID NO: 2976-3042, SEQ ID NO: 3046-3268, and any combination thereof, or (ii) a ribonucleoprotein (RNP) complex comprising Cas protein and one or more gRNA molecules comprising a sequence comprising at least 85% sequence identity to at least one of SEQ ID NO: 4-219, SEQ ID NO: 223-453, SEQ ID NO: 457-673, SEQ ID NO: 677-1013, SEQ ID NO: 1017-1304, SEQ ID NO: 1308-1490, SEQ ID NO: 1494-1797, SEQ ID NO: 1801-2089, SEQ ID NO: 2633-2809, SEQ ID NO: 2813-2972, SEQ ID NO: 2976-3042 or SEQ ID NO: 3046-3268, and any combination thereof, to modify the phytoestrogen content of the Soybean plant, plant part, or plant cell, or (b) (i) a nucleotide sequence encoding a Cas molecule and one or more gRNA molecules comprising a sequence selected from the group consisting of a sequence comprising at least 85% sequence identity to at least one of SEQ ID NO: 2093-2199, SEQ ID NO: 2203-2295, SEQ ID NO: 2299-2629, and any combination thereof, or (ii) a ribonucleoprotein (RNP) complex comprising Cas protein and one or more gRNA molecules comprising a sequence comprising at least 85% sequence identity to at least one of SEQ ID NO: 2093-2199, SEQ ID NO: 2203-2295, SEQ ID NO: 2299-2629, and any combination thereof, respectively, to modify the protein content of the Soybean plant, plant part, or plant cell.

75-77. (canceled)
